(19) United States
(12) Patent Application Publication
(10) Pub. No.: US 2003/0195707 A1
(43) Pub. Date: Oct. 16, 2003

(54) Methods of dna marker-based genetic analysis using estimated haplotype frequencies and uses thereof

(76) Inventors: Nicholas J Schork, San Diego (CA); Margaret Daniele Fallin, Baltimore, MD (US); Sebastien Lissarrague, Paris (FR)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/296,867

(22) PCT Filed: May 22, 2001
(86) PCT No.: PCT/IB01/01284

(51) Int. Cl. G06F 19/00; G01N 33/48; G01N 33/50

(52) U.S. Cl. 702/20

(57) ABSTRACT

The present invention is primarily drawn to methods of DNA marker-based genetic analysis using estimated haplo-type frequencies to draw inferences about the relationship between haplotypes and traits or diseases. Unlike many haplotype analysis methods that require phase information that can be difficult to obtain from samples of non-haploid species, the instant methods are based on strategies for estimating haplotype frequencies from unphased diploid genotype data using the Estimation-Maximization (E-M) algorithm to overcome the missing phase information. These estimated haplotype frequencies can then be used in a variety of statistical analyses, including those to infer the existence of a disease gene. The process can include: 1) estimating haplotype frequencies; 2) computing test statistics; and 3) drawing inferences.

### RELATED APPLICATIONS

This application claims priority from provisional application No. 60/207,904 filed on May 25, 2000 and also from provisional application No. 60/221,850, filed on Jul. 28, 2000.

### FIELD OF THE INVENTION

The present invention relates to applied statistical genomics, and is primarily drawn to methods of DNA marker-based genetic analysis using estimated haplotype frequencies to draw inferences about the relationship between haplotypes and disease.

### BACKGROUND OF THE INVENTION

The following discussion is meant to aid in the understanding of the invention, but is not intended to, and is not admitted to, describe prior art to the invention.

Humans are a diploid species; they inherit two copies of each of their 23 chromosomes, one from the mother and one from the father. Most modern genotyping protocols, however, focus on the determination of variants or alleles possessed by an individual at specific genetic loci (i.e., genotype). They do not provide information as to which variants or alleles were transmitted together on the same chromosome from each parent (i.e., haplotype). Thus, most genotyping protocols result purely in genotype information; they produce information about the pair of alleles an individual possesses at each locus, but not necessarily haplotype information which would reveal the alleles that have been inherited together on the same paternal or maternal chromosome.

A lack of haplotype information complicates genetic analyses and gene mapping initiatives since without explicit haplotype information, there is ambiguity with respect to the origin of alleles at neighboring loci. For example, it is difficult to determine if there are differences in the frequency of certain haplotypes between individuals with a disease (‘cases’) and individuals without the disease (‘controls’) in the absence of haplotype information.

Haplotype information can be obtained in different ways, including: 1) genotyping parents and other relatives of a target individual and then inferring “phase” or the likely distribution of alleles on maternal and paternal chromosomes transmitted to the target individual, and 2) using molecular laboratory techniques, such as long-range PCR (Clark et al., American Journal of Human Genetics 63, 595-612 (1998)) that can directly produce haplotype information. However, both these techniques are costly and at times difficult or impossible to implement (e.g., a target individual may not have any accessible relatives).

As the methods for polymorphism discovery and mass genotyping continue to provide enormous amounts of data for the investigation of genetic variation and its relationship to phenotypic variation (Chakravarti, Nature Genetics 19, 216-217 (1998)) the challenge shifts to the development of methods that best utilize this wealth of information, including valid haplotype estimation and statistical tests that incorporate these estimates. Characterizing the relationships between genotypic and phenotypic variation can provide important information regarding the etiology and pathogenesis of common diseases, which can in turn help elucidate new target pathways and molecules, yielding new approaches to treatment and prevention therapies.

Characterization of genetic risk, independently and/or interactively with environmental backgrounds, can also improve the prediction, diagnosis, and prognosis of disease in an individual, allowing efficient targeting of preventative measures, and contributing to more informative genetic counseling. At the population level, determination of disease predisposing gene frequencies and penetrances can also enable more efficient allocation of resources guided by the estimated public-health impact of particular genes in the population at large.

However, there is currently a debate concerning three related sets of issues. First, there is a lack of consensus as to the best way to use high-density single nucleotide polymorphism (SNP) maps to identify complex disease genes in large, freely-mixing populations. For example, some researchers advocate the use of simple family-based single-locus association studies (Risch, et al. Science 273, 1516-1517 (1996)). Others argue that linkage analyses, rather than association analyses, will be the most appropriate for use in such populations, given the possible allelic heterogeneity underlying complex diseases and the likely insufficient marker density of near-future high-resolution maps (Terwilliger, et al, Current Opinion in Biotechnology, 9, 578-594 (1998)); Kruglyak, Nature Genetics 17, 21-24 (1997)). Finally, others argue that high-resolution SNP mapping may be so fraught with statistical difficulties, such as the preservation of reasonable false positive rates and power, that it may be better to focus on candidate gene analyses or the use of other sorts of markers besides SNPs (Chapman et al, American Journal of Human Genetics, 63, 1872-1885 (1998)).

A second issue is that there is simply a lack of published empirical data attesting to the utility (or lack thereof of SNP-based association studies in large populations. For example, it is unclear whether or not the strength of linkage disequilibrium (LD) between putative trait-influencing alleles and neighboring marker locus alleles in large, freely-mixing populations is sufficient enough to support LD-based association analysis with anonymous SNPs and non-family-based sampling units such cases and controls (Terwilliger, et al, Current Opinion in Biotechnology, 9, 578-594 (1998)); Clark, et al. American Journal of Human Genetics 63,595-612 (1998); Chakravarti, Nature Genetics 19, 216-217 (1998)).

In addition, it is also unclear whether or not the effects of admixture and stratification in large populations for which case/control sampling might be undertaken for an association study will be pronounced enough to cause increased false positive results or confound the detection of true positives. Finally, it is arguable that variation in relevant genes that actually influence phenotypic expression may be so large as to preclude detection of simple associations between particular variants and disease (Terwilliger, et at, Current Opinion in Biotechnology, 9, 578-594 (1998); Chakravarti, Nature Genetics 19, 216-217 (1998)).

A third issue is that relevant analyses should focus on the transmission of multilocus haplotypes, as opposed to alleles at individual loci, to fully exploit high-density maps. The identification and study of the transmission of haplotypes, however, requires knowledge of phase information in the individuals studied. Methods for determining phase and assigning haplotypes usually require either laborious chromosome isolation or other laboratory-based strategies or genotypic information on relatives of the individuals studied. Thus, analysis of unrelated individuals, as in case/control studies where simple genotypic data is collected, is problematic.

Estimation of quantities, such as haplotype frequencies, from data in which only some individuals in the sample have complete information can be accomplished through statistical algorithms such as the E-M algorithm (Excoffier et at, Molecular Biology and Evolution, 12, 921-927 (1995); Hawley et at, Journal of Heredity, 86, 409-411 (1995); Long et at, American Journal of Human Genetics, 56, 799-810 (1995). The E-M algorithm and related algorithms use haplotype frequencies from unambiguous individuals to project and infer haplotypes for the ambiguous individuals.

The E-M algorithm first computes expected genotype probabilities based on haplotype frequency estimates provided by genotype data from individuals with complete information and projected frequency information for individuals that have ambiguous genotypes. This is the ‘expectation’ step. Once estimates of the frequencies are obtained, the probability of each possible pair of haplotypes for each individual's genotype configuration is computed. These probabilities provide information about how compatible the estimated haplotype frequencies are with the genotype data. This step is the ‘maximization’ step. These two steps are pursued in sequence until the estimates converge (i.e., do not change with subsequent expectation and maximization calculations).

Currently available software programs allow the estimation of haplotype frequencies for multiple allele systems (Excoffier et al., Microbiology & Evolution 12, 921-927 (1995); Long et al. Am. J. Hum Gen. 56, 799-810 (1995); Hawley et at, Journal of Heredity, 86, 409411 (1995)), and as a result are computationally inefficient and impractical for doing large association studies. In addition, these programs are not designed to automatically repeat the maximization process, which may result in a convergence at a local rather than the desired global maximum. Further, these programs do not permit statistical inference drawing among groups.

### SUMMARY OF THE INVENTION

The invention is drawn, inter alia, to a significantly improved method and software program that is optimized for use with SNPs or any other 2 allele system, rather than for use with multiple allele systems and is designed to automatically repeat the maximization process to achieve convergence at a global maximum. This system is significantly faster and more efficient than any of the currently available software programs and thus permits the several thousand analyses necessary for doing association studies for clinical trials, for example. In addition, the method and software program of the invention is also designed for statistical inference drawing among groups, a feature important for the interpretation of results.

Embodiments of the invention relate to systems and methods for overcoming the lack of phase, or lack of haplotype information, in a sample of individuals by estimating haplotype frequencies from the genotype data collected on each individual in a sample. The estimated haplotype frequencies are then used in a variety of statistical analyses, including those to infer the statistical significance between SNPs in case and control data for clinical trials, drug tests, disease gene association studies, and association studies with other phenotypic markers of disease, such as levels of a protein of interest in the serum.

One embodiment of the process includes one or more of the following steps: 1) estimating the haplotype frequencies of individuals in case (e.g., disease) and control (e.g., non-disease) groups; 2) computing a test statistic to assess the difference in the estimated frequencies of the haplotypes between diseased and non-diseased individuals, for example; and 3) estimating the significance of the test statistic to facilitate drawing appropriate inferences.

Described herein is a suite of computer-based analytic methodologies for assessing the association between multiple Single Nucleotide Polymorphisms (SNPs) within a defined genomic region and a disease assuming simple case/control samples and genotype data. These methods include an Estimation-Maximization (E-M) algorithm that estimates haplotype frequencies from SNP data. Embodiments of the invention also provide statistical methods for Linkage Disequilibrium (LD) mapping and candidate gene analyses, as well as general population comparisons, based on the resulting estimated haplotype frequencies. These methods take advantage of estimated haplotype frequencies in each of the case and control groups and simulation-based tests of relevant hypotheses.

The accuracy of the haplotype estimation methods described herein have been assessed as discussed below. The methods accommodate many computational problems thought to plague the use of the E-M algorithm, such as a potential for convergence to local maxima. The E-M algorithm was found to produce accurate haplotype frequency estimates, even for biallelic loci with alleles departing from equilibrium. Many factors that may influence accuracy can be assessed empirically within a data set—a fact which can be used create ‘diagnostics’ that a user can turn to for assessing potential inaccuracies in estimation.

In one embodiment, the invention is drawn to a method for analyzing genetic data that includes haplotype estimation, analysis using test statistics, and inference drawing. Haplotype estimation is performed using either a laboratory data-based estimate of haplotype frequencies, or an E-M algorithm based estimate. The E-M algorithm-based estimate can be performed using a computer program such as Arlequin (Schneider et al. Genetics and Biometry Laboratory, University of Geneva, Switzerland (2000) anthropologue.unige.ch/arlequin), or any other method that uses E-M to estimate haplotype frequencies. Analysis using test statistics can be performed through logistic regression, other regression-based tests, individual haplotype tests, or preferably omnibus test statistics. The inference drawing can be based on asymptotic tests, deriving exact distributions of relevant quantities, empirical distributions of relevant quantities, parametric bootstrap tests, nonparametric bootstrap, or more preferably randomization tests. The genetic data that can be analyzed using these methods includes, but is not limited to, SNP case and control data for clinical trials, drug tests, disease gene, association studies, and association studies with other phenotypic markers of disease, such as levels of a protein of interest in the serum.

In a second aspect, the invention is drawn to a computer program that performs the method described in the first aspect.

In a third aspect, the invention is drawn to a method for estimating haplotypes using a computer software program of the invention.

In a fourth aspect, the invention is drawn to a method of genetic analysis using the omnibus test statistic of the invention.

In a fifth aspect, the invention is drawn to a computer program that performs the method described in the fourth aspect.

In a sixth aspect, the invention is drawn to a method of determining the statistical significance of a difference between haplotype frequency profiles between at least two groups of individuals comprising: determining the combined likelihood that said at least two groups of individuals are derived from the same distribution of haplotypes; determining the sum of the separate likelihoods that each of said at least two groups of individuals are derived from the same distribution of haplotypes; determining the difference of said sum and said combined likelihood; and

determining the significance of this difference by simulating hypothetical groups by randomly permuting the haplotypes between groups to determine the probability that the groups do not come from the same distribution of haplotypes. In preferred embodiments, the method further comprises calculating all possible single-haplotype chi-square tests prior to said determining significance, and/or further comprises a method of assessing the statistical significance of individual haplotypes using an odds ratio or a P-excess value. In some preferred embodiments, this method is a computer program.

In a seventh aspect, the invention features a system for determining the statistical significance of the difference between haplotype frequency profiles of at least two groups of individuals, comprising: first instructions for determining the combined likelihood that said at least two groups of individuals are derived from the same distribution of haplotypes second instructions for determining the sum of the separate likelihoods that each of said at least two groups of individuals are derived from the same distribution of haplotypes; third instructions for determining the difference of said sum and said combined likelihood; and fourth instructions for determining the significance of this difference by simulating hypothetical groups by randomly permuting the haplotypes between groups to determine the probability that the groups do not come from the same distribution of haplotypes. In preferred embodiments, the computer system further comprises fifth instructions for calculating all possible single-haplotype chi-square tests prior to said determining significance, and/or further comprises fifth instructions for a method of assessing the statistical significance of individual haplotypes using an odds ratio or a P-excess value.

In an eighth aspect, the invention features a programmed storage device comprising instructions that when executed perform a method comprising: determining the determining the statistical significance of the difference between haplotype frequency profiles of at least two groups of individuals comprising comparing the final likelihood that all groups come from the same distribution of haplotypes with the sum of the final likelihoods for each group separately; and determining the significance of this difference by simulating hypothetical groups by randomly permuting the haplotypes between groups to determine the probability that the groups do not come from the same distribution of haplotypes. In preferred embodiments, the programmed storage device further comprises instructions that when executed perform a method of calculating all possible single-haplotype chi-square tests prior to said determining significance, and/or further comprises instructions that when executed perform a method of assessing the statistical significance of individual haplotypes using an odds ratio or a P-excess value. In some preferred embodiments the instructions are on a computer-readable medium.

In a ninth aspect, the invention features a method of determining the statistical significance of the difference between haplotype frequency profiles of at least two groups of individuals, comprising: estimating haplotype frequencies using single nucleotide polymorphism data for each group individually and in combination with the other group, wherein all haplotype and diplotype probabilities are calculated once and are stored, and wherein the maximization process is automatically repeated using random starting values. In preferred embodiments, all haplotypes are coded with binary mask arrays, and wherein identical genotypes are grouped prior to performing operations. In some preferred embodiments, this method is a computer program.

In a tenth aspect, the invention features a computer system for determining the statistical significance of the difference between haplotype frequency profiles of at least two groups of individuals, comprising: instructions that when executed perform the method of estimating haplotype frequencies using single nucleotide polymorphism data for each group individually and in combination with the other group, wherein all haplotype and diplotype probabilities are calculated once and are stored, and wherein the maximization process is automatically repeated using random starting values. In preferred embodiments, all haplotypes are coded with binary mask arrays, and wherein identical genotypes are grouped prior to performing operations.

In an eleventh aspect, the invention features a programmed storage device comprising instructions that when executed perform the method of: determining the statistical significance of the difference between haplotype frequency profiles of at least two groups of individuals, comprising estimating haplotype frequencies using single nucleotide polymorphism data for each group individually and in combination with the other group, wherein all haplotype and diplotype probabilities are calculated once and are stored, and wherein the maximization process is automatically repeated using random starting values. In preferred embodiments, all haplotypes are coded with binary mask arrays, and wherein identical genotypes are grouped prior to performing operations. In some preferred embodiments the instructions are on a computer-readable medium.

In a twelfth aspect, the invention features a method of determining the statistical significance of the difference between haplotype frequency profiles of at least two groups of individuals, comprising:estimating haplotype frequencies using single nucleotide polymorphism data for each group individually and in combination with the other group, wherein all haplotype and diplotype probabilities are calculated once and are stored, and wherein the maximization process is automatically repeated using random starting values, to determine final likelihoods; comparing the final likelihood that all groups come from the same distribution of haplotypes with the sum of the final likelihoods for each group separately; and determining the significance of this difference by simulating hypothetical groups by randomly permuting the haplotypes between groups to determine the probability that the groups do not come from the same distribution of haplotypes. In preferred embodiments, all haplotypes are coded with binary mask arrays, and wherein identical genotypes are grouped prior to performing operations. In some preferred embodiments, this method is a computer program.

In a thirteenth aspect, the invention features a computer system for determining the statistical significance of the difference between haplotype frequency profiles of at least two groups of individuals, comprising: first instructions that when executed perform the method of estimating haplotype frequencies using single nucleotide polymorphism data for each group individually and in combination with the other group, wherein all haplotype and diplotype probabilities are calculated once and are stored, and wherein the maximization process is automatically repeated using random starting values, to determine final likelihoods; second instructions for comparing the final likelihood that all groups come from the same distribution of haplotypes with the sum of the final likelihoods for each group separately; and third instructions for determining the significance of this difference by simulating hypothetical groups by randomly permuting the haplotypes between groups to determine the probability that the groups do not come from the same distribution of haplotypes. In preferred embodiments, all haplotypes are coded with binary mask arrays, and wherein identical genotypes are grouped prior to performing operations.

In a fourteenth aspect, the invention features a programmed storage device comprising instructions that when executed perform a method determining the statistical significance of the difference between haplotype frequency profiles of at least two groups of individuals, comprising: a first module adapted to perform a method of estimating haplotype frequencies using single nucleotide polymorphism data for each group individually and in combination with the other group, wherein all haplotype and diplotype probabilities are calculated once and are stored, and wherein the maximization process is automatically repeated using random starting values, to determine final likelihoods; a second module adapted to compare the final likelihood that all groups come from the same distribution of haplotypes with the sum of the final likelihoods for each group separately; and a third module adapted to determine the significance of this difference by simulating hypothetical groups by randomly permuting the haplotypes between groups to determine the probability that the groups do not come from the same distribution of haplotypes. In preferred embodiments, all haplotypes are coded with binary mask arrays, and wherein identical genotypes are grouped prior to performing operations. In some preferred embodiments the instructions are on a computer-readable medium.

In a fifteenth aspect, the invention features a method of detecting an association between a haplotype and a phenotype, comprising: estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine final likelihoods; comparing the final likelihood that both groups come from the same distribution of haplotypes with the sum of the final likelihoods for each group separately; and determining the significance of this difference by simulating hypothetical groups by randomly permuting the haplotypes between groups to determine the probability that the groups do not come from the same distribution of haplotypes and determine whether a statistically significant association exists between said haplotype and said phenotype. In some preferred embodiments, this method is a computer program.

In a sixteenth aspect, the invention features a method of detecting an association between a haplotype and a phenotype, comprising: estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine whether a statistically significant association exists between said haplotype and said phenotype. In some preferred embodiments, this method is a computer program.

In a seventeenth aspect, the invention features a method of detecting an association between a haplotype and a phenotype, comprising: comparing the final likelihood that the members of an affected and an unaffected group come from the same distribution of haplotypes with the sum of the final likelihoods for each group separately; determining the significance of this difference by simulating hypothetical groups by randomly permuting the haplotypes between groups to determine the probability that the groups do not come from the same distribution of haplotypes and whether a statistically significant association exists between said haplotype and said phenotype. In some preferred embodiments, this method is a computer program.

In an eighteenth aspect, the invention features a system for detecting an association between a haplotype and a phenotype, comprising: first instructions for estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine final likelihoods; second instructions for comparing the final likelihood that both groups come from the same distribution of haplotypes with the sum of the final likelihoods for each group separately; and third instructions for determining the significance of this difference by simulating hypothetical groups by randomly permuting the haplotypes between groups to determine the probability that the groups do not come from the same distribution of haplotypes and determine whether a statistically significant association exists between said haplotype and said phenotype.

In a nineteenth aspect, the invention features a system for detecting an association between a haplotype and a phenotype, comprising: instructions for estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine whether a statistically significant association exists between said haplotype and said phenotype.

In a twentieth aspect, the invention features a system for detecting an association between a haplotype and a phenotype, comprising: first instructions for comparing the final likelihood that the members of an affected and an unaffected group come from the same distribution of haplotypes with the sum of the final likelihoods for each group separately; second instructions for determining the significance of this difference by simulating hypothetical groups by randomly permuting the haplotypes between groups to determine the probability that the groups do not come from the same distribution of haplotypes and whether a statistically significant association exists between said haplotype and said phenotype.

In a twenty-first aspect, the invention features a programmed storage device comprising instructions that when executed perform a method of detecting an association between a haplotype and a phenotype, comprising: estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine final likelihoods; comparing the final likelihood that both groups come from the same distribution of haplotypes with the sum of the final likelihoods for each group separately; and determining the significance of this difference by simulating hypothetical groups by randomly permuting the haplotypes between groups to determine the probability that the groups do not come from the same distribution of haplotypes and determine whether a statistically significant association exists between said haplotype and said phenotype. In some preferred embodiments the instructions are on a computer-readable medium.

In a twenty-second aspect, the invention features a programmed storage device comprising instructions that when executed perform a method of detecting an association between a haplotype and a phenotype, comprising: estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine whether a statistically significant association exists between said haplotype and said phenotype. In some preferred embodiments the instructions are on a computer-readable medium.

In a twenty-third aspect, the invention features a programmed storage device comprising instructions that when executed perform a method of detecting an association between a haplotype and a phenotype, comprising: comparing the final likelihood that the members of an affected and an unaffected group come from the same distribution of haplotypes with the sum of the final likelihoods for each group separately; determining the significance of this difference by simulating hypothetical groups by randomly permuting the haplotypes between groups to determine the probability that the groups do not come from the same distribution of haplotypes and whether a statistically significant association exists between said haplotype and said phenotype. In some preferred embodiments the instructions are on a computer-readable medium.

In a twenty-fourth aspect, the invention features a computer-readable data signal embedded in a transmission medium that when executed performs a method of determining the statistical significance of the difference between haplotype frequency profiles of at least two groups of individuals, comprising code segments comparing the final likelihood that all groups come from the same distribution of haplotypes with the sum of the final likelihoods for each group separately; and code segments determining the significance of this difference by simulating hypothetical groups by randomly permuting the haplotypes between groups to determine the probability that the groups do not come from the same distribution of haplotypes. In preferred embodiments, the computer-readable data signal further comprises instructions that when executed perform a method of calculating all possible single-haplotype chi-square tests prior to said determining significance, and/or further comprises instructions that when executed perform a method of assessing the statistical significance of individual haplotypes using an odds ratio or a P-excess value.

In a twenty-fifth aspect, the invention features a computer-readable data signal embedded in a transmission medium that when executed performs a method of determining the statistical significance of the difference between haplotype frequency profiles of at least two groups of individuals, comprising code segments estimating haplotype frequencies using single nucleotide polymorphism data for each group individually and in combination with the other group, wherein all haplotype and diplotype probabilities are calculated once and are stored, and wherein the maximization process is automatically repeated using random starting values. In preferred embodiments, all haplotypes are coded with binary mask arrays, and wherein identical genotypes are grouped prior to performing operations.

In a twenty-sixth aspect, the invention features a computer-readable data signal embedded in a transmission medium that when executed performs a method determining the statistical significance of the difference between haplotype frequency profiles of at least two groups of individuals, comprising code segments adapted to perform a method of estimating haplotype frequencies using single nucleotide polymorphism data for each group individually and in combination with the other group, wherein all haplotype and diplotype probabilities are calculated once and are stored, and wherein the maximization process is automatically repeated using random starting values, to determine final likelihoods; code segments adapted to compare the final likelihood that all groups come from the same distribution of haplotypes with the sum of the final likelihoods for each group separately; and code segments adapted to determine the significance of this difference by simulating hypothetical groups by randomly permuting the haplotypes between groups to determine the probability that the groups do not come from the same distribution of haplotypes. In preferred embodiments, all haplotypes are coded with binary mask arrays, and wherein identical genotypes are grouped prior to performing operations.

In a twenty-seventh aspect, the invention features a computer-readable data signal embedded in a transmission medium that when executed performs a method of detecting an association between a haplotype and a phenotype, comprising code segments estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine final likelihoods; code segments comparing the final likelihood that both groups come from the same distribution of haplotypes with the sum of the final likelihoods for each group separately; and code segments determining the significance of this difference by simulating hypothetical groups by randomly permuting the haplotypes between groups to determine the probability that the groups do not come from the same distribution of haplotypes and determine whether a statistically significant association exists between said haplotype and said phenotype.

In a twenty-eighth aspect, the invention features a computer-readable data signal embedded in a transmission medium that when executed performs a method of detecting an association between a haplotype and a phenotype, comprising code segments estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine whether a statistically significant association exists between said haplotype and said phenotype.

In a twenty-ninth aspect, the invention features a computer-readable data signal embedded in a transmission medium that when executed performs a method of detecting an association between a haplotype and a phenotype, comprising code segments comparing the final likelihood that the members of an affected and an unaffected group come from the same distribution of haplotypes with the sum of the final likelihoods for each group separately; code segments determining the significance of this difference by simulating hypothetical groups by randomly permuting the haplotypes between groups to determine the probability that the groups do not come from the same distribution of haplotypes and whether a statistically significant association exists between said haplotype and said phenotype.

### BRIEF DESCRIPTION OF THE DRAWINGS

FIG. 1 is an overall block diagram of one embodiment of the invention, beginning with haplotype estimation, continuing through use of a test statistic and ending after an inference drawing procedure.

FIG. 2 is a block diagram of one embodiment of an automated system.

FIG. 3 is a flow diagram of one embodiment of a process of estimating haplotype frequencies from DNA marker genetic data.

FIG. 4 is a flow diagram of one embodiment of a process for estimating haplotype frequencies of cases, controls, and combined cases/controls.

FIG. 5 is a flow diagram of one embodiment of a process for testing the significance of differences between haplotype frequencies.

FIG. 6 is a block diagram illustrating the conceptual framework for simulation studies and accuracy comparisons.

FIGS. 7A-C are graphs showing the distribution of maximum log-likelihoods from the estimation procedure as a function of algorithm settings: convergence criterion (FIG. 7A), maximum iterations (FIG. 7B), and number of restarts at different random initial frequency values (FIG. 7C). For these analyses, 500 data sets of size 200 were simulated for a 5-locus system (mean frequency=0.03125, variance=10.0). The above analyses were performed on the same 500 simulated sets each time, with the setting of interest progressively adjusted to a more stringent value.

FIGS. 8a and 8b are line graphs showing the accuracy of program estimates as a function of sample size. Average MSE (a) and |Bias| (b) computer over 500 simulated data sets assuming 5-locus system.

FIGS. 9a and 9b are line graphs showing the accuracy of program estimates as a function of the frequency of lack of ambiguity in genotype data in the sample. The x axis indicates the proportion of homozygous loci across all individuals and loci from MSE (a) and |Bias| (b) are plotted. The above analyses are based on 1000 simulated sets of size 200.

FIGS. 10a and 10b are line graphs showing the accuracy of program estimates as a function of the frequency of the most common haplotype in the sample. The x axis indicates the frequency of the most common estimated haplotype across the simulated data sets. MSE (a) and |Bias| (b) are plotted. The analyses are based on 1000 simulated sets of size 200 for a 5-locus system.

FIGS. 11a and 11b are line graphs showing the accuracy of program estimates as a function of the minor allele frequency across all loci. MSE (a) and |Bias| (b) are plotted. The analyses are based on 1000 simulated sets of size 200 for a 5-locus system.

FIG. 12 is a line graph showing the accuracy of program estimates as a function of the average chi-squared value for HWE tests across all loci. The y axis indicates MSE between final haplotype frequency estimates and sample set values or simulating parameter values. The analyses are based on 1000 simulated sets of size 200 for a 5-locus system.

FIGS. 13a and 13b are line graphs showing the accuracy of program estimates as a function of the number of loci used to construct haplotypes (2, 3, 4, 5, 7, 10 locus systems were studied). MSE (a) and |Bias| (b) are plotted. The analyses are based on 1000 simulated sets of size 200.

FIG. 14 is a table depicting the Regression of absolute value of bias between estimated and generating haplotype frequencies on all factors.

FIG. 15 is a table containing haplotype frequency estimates and significance levels of case-control comparison from permutation tests.

FIGS. 16A-D are bar graphs showing the frequency histograms of the omnibus test statistics resulting from 1000 permutations of case and control status for the four-locus haplotypes which include the APOE ε4 allele locus: markers 1, 3, 4, and 6 (panel A) and four-locus haplotypes which only include SNPs that flank the APOE c4 allele locus and the locus in strong disequilibrium with it: markers 1, 2, 5, and 6 (panel B). Panel C shows the empirical distribution for the four-locus system on ch. 19 that does not contain ε4 allele locus or SNPs which flank the e4 locus: markers 5, 6, 7, and 8. Panel D shows the empirical distribution for a four-locus system on chromosome 13: markers _{c13}2, _{c13}3, _{c13}4, and _{c13}5. The positions of the test statistics computed from the actual data relative to the estimated distribution are also provided.

### DETAILED DESCRIPTION OF THE INVENTION

Definitions

A computer-readable medium includes any media that a computer can read, including but not limited to, CD, floppy disk, hard-drive, magneto-optical, tape drive, zip drive, punch cards, Read Only Memory (ROM), Random Access Memory (RAM), other memory devices, propagated data signals, and paper (scanned, for example).

A database includes indexed and freeform tables for storing data. Within each table are a series of fields that store data strings, such as names, addresses, chemical names, and the like. However, it should be realized that several types of databases are available. For example, a database might only include a list of data strings arranged in a column. Other databases might be relational databases wherein several two dimensional tables are linked through common fields. Embodiments of the invention are not limited to any particular type of database.

An input device can be, for example, a keyboard, rollerball, mouse, voice recognition system, automated script from another computer that generates a file, or other device capable of transmitting information from a customer to a computer. The input device can also be a touch screen associated with the display, in which case the customer responds to prompts on the display by touching the screen. The customer may enter textual information through the input device such as the keyboard or the touch-screen.

Instructions refer to computer-implemented steps for processing information in the system. Instructions can be implemented in software, firmware or hardware and include any type of programmed step undertaken by components and modules of the system.

One example of a Local Area Network may be a corporate computing network, including access to the Internet, to which computers and computing devices comprising the system are connected. In one embodiment, the LAN conforms to the Transmission Control Protocol/Internet Protocol (TCP/IP) industry standard. In alternative embodiments, the LAN may conform to other network standards, including, but not limited to, the International Standards Organization's Open Systems Interconnection, IBM's SNA, Novell's Netware, and Banyan VINES.

A microprocessor as used herein may be any conventional general purpose single- or multi-chip microprocessor such as a Pentium® processor, a Pentium® Pro processor, a 8051 processor, a MIPS® processor, a Power PC® processor, or an ALPHA® processor. In addition, the microprocessor may be any conventional special purpose microprocessor such as a digital signal processor or a graphics processor. The microprocessor typically has conventional address lines, conventional data lines, and one or more conventional control lines.

A programmed storage device is any computer readable media on which a program readable by a computer has been stored. Stored refers to both brief elements of time (measured in seconds or less) and log elements of time (seconds and more up to years).

A propagated signal refers to the transmission of programs or data structures through transmission media. Transmission media can include, but is not limited to, the internet, modems, telephone lines, cable, fiber optic, and laser.

A code segment is an area of computer memory that contains assembly language instructions for performing specific tasks.

The system is comprised of various modules as discussed in detail below. As can be appreciated by one of ordinary skill in the art, each of the modules comprises various sub-routines, instructions, commands, procedures, definitional statements and macros. Each of the modules are typically separately compiled and linked into a single executable program. Therefore, the following description of each of the modules is used for convenience to describe the functionality of the preferred system. Thus, the processes that are undergone by each of the modules may be arbitrarily redistributed to one of the other modules, combined together in a single module, or made available in, for example, a shareable dynamic link library.

The system may include any type of electronically connected group of computers including, for instance, the following networks: Internet, Intranet, Local Area Networks (LAN) or Wide Area Networks (WAN). In addition, the connectivity to the network may be, for example, remote modem, Ethernet (IEEE 802.3), Token Ring (IEEE 802.5), Fiber Distributed Datalink Interface (FDDI) or Asynchronous Transfer Mode (ATM). Note that computing devices may be desktop, server, portable, hand-held, set-top, or any other desired type of configuration. As used herein, an Internet includes network variations such as public internet, a private internet, a secure internet, a private network, a public network, a value-added network, an intranet, and the like.

The system may be used in connection with various operating systems such as: UNIX, Disk Operating System (DOS), OS/2, Windows 3.X, Windows 95, Windows 98, Windows 2000 and Windows NT.

The various software aspects of the system may be written in any programming language such as C, C++, BASIC, Pascal, Perl, Java, and FORTRAN and ran under the well-known operating system. C, C++, BASIC, Pascal, Java, and FORTRAN are industry standard programming languages for which many commercial compilers can be used to create executable code.

A system preferably includes one or more computers and associated peripherals that carry out selected functions. For example, a User system preferably includes the computer hardware, software and firmware for executing the specific software instructions described below. A system should not be interpreted as being limited to be a single computer or microprocessor, and may include a network of computers, or a computer having multiple microprocessors.

Transmission Control Protocol (TCP) is a transport layer protocol used to provide a reliable, connection-oriented, transport layer link among computer systems. The network layer provides services to the transport layer. Using a two-way handshaking scheme, TCP provides the mechanism for establishing, maintaining, and terminating logical connections among computer systems. TCP transport layer uses IP as its network layer protocol. Additionally, TCP provides protocol ports to distinguish multiple programs executing on a single device by including the destination and source port number with each message. TCP performs functions such as transmission of byte streams, data flow definitions, data acknowledgments, lost or corrupt data re-transmissions and multiplexing multiple connections through a single network connection. Finally, TCP is responsible for encapsulating information into a datagram structure.

The term “allele” is used herein to refer to variants of a nucleotide sequence. A biallelic polymorphism has two forms. Diploid organisms may be homozygous or heterozygous for an allelic form.

The term “biallelic polymorphism” and “biallelic marker” are used interchangeably herein to refer to a single nucleotide polymorphism (SNP) having two alleles at a fairly high frequency in the population. A “biallelic marker allele” refers to the nucleotide variants present at a biallelic marker site. Typically, the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker”.

The term “diplotype” as used herein refers to the identity of the alleles on both chromosomes in an individual.

The term “genotype” as used herein refers the identity of the alleles present in an individual or a sample. In the context of the present invention, a genotype preferably refers to the description of the biallelic marker alleles present in an individual or a sample.

The term “genotyping” a sample or an individual for a biallelic marker involves determining the specific allele or the specific nucleotide carried by an individual at a biallelic marker.

The term “haplotype” refers to a combination of alleles present in an individual or a sample. In the context of the present invention, a haplotype preferably refers to a combination of biallelic marker alleles found in a given individual and which may be associated with a phenotype. Haplotype typically refers to sets of alleles on the same chromosomal segment. Haplotypes tend to be transmitted as a block from generation to generation.

The term “heterozygosity rate” is used herein to refer to the incidence of individuals in a population that are heterozygous at a particular allele. In a biallelic system, the heterozygosity rate is on average equal to 2Pₐ(1−Pₐ), where Pₐ is the frequency of the least common allele. In order to be useful in genetic studies, a genetic marker should have an adequate level of heterozygosity to allow a reasonable probability that a randomly selected person will be heterozygous.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A single nucleotide polymorphism is the replacement of one nucleotide by another nucleotide at the polymorphic site. Deletion of a single nucleotide or insertion of a single nucleotide also gives rise to single nucleotide polymorphisms. In the context of the present invention, “single nucleotide polymorphism” preferably refers to a single nucleotide substitution. Typically, between different individuals, the polymorphic site may be occupied by two different nucleotides.

SNPs as used herein, refer to biallelic markers, which are genome-derived polynucleotides that exhibit biallelic polymorphism. As used herein, the term biallelic marker means a biallelic single nucleotide polymorphism. As used herein, the term polymorphism may include a single base substitution, insertion, or deletion. By definition, the lowest allele frequency of a biallelic polymorphism is 1% (sequence variants which show allele frequencies below 1% are called rare mutations or ideomorphs). There are potentially more than 10⁷ biallelic markers that can easily be typed by routine automated techniques, such as sequence- or hybridization-based techniques, out of which 10⁶ are sufficiently informative for mapping purposes.

The terms “trait” and “phenotype” are used interchangeably herein and refer to any visible, detectable or otherwise measurable property of an organism such as symptoms of, or susceptibility to a disease for example. Typically the terms “trait” or “phenotype” are used herein to refer to symptoms of, or susceptibility to a disease, a beneficial response to or side effects related to a treatment.

Statistical significance, is used herein as it is typically used by those with skill in the art. It is a measure of the probability that an observed difference would have been observed simply by chance and is not the result of a “real” difference between two groups, for example. Thus the lower the probability that the observed difference would have happened by chance, the less likely that it happened by chance. Statistical significance is based on p-values. A p-value <0.05 is typically considered statistically significant, although in some instances a p-value of <0.01 or even <0.005 or <0.001 is preferred. In general, the lower the p-value, the less likely that an observed difference occurred by chance, and thus, the more statistically significant the difference.

### Overview

One embodiment of the invention provides a process for estimating haplotypes from genotype and SNP data, and using the estimated haplotypes to make inferences about the linkage between a particular haplotype and a disease state. This process preferably includes: 1) Estimating the haplotype frequencies; 2) Computing a test statistic to assess the difference in the estimated frequencies of the haplotypes between two groups (diseased (cases) and non-diseased (controls) individuals, for example); and 3) Determining the significance of the test statistic to facilitate drawing appropriate inferences.

1. Estimation of Haplotype Frequencies

The estimation of haplotype frequencies from genotype data gathered on a sample of individuals is based on the fact that the haplotypes of some individuals in the sample are unambiguous. This allows the ambiguous haplotypes to be estimated using statistical predictions. Individuals that are unambiguous with respect to phase or haplotype information have homozygous genotypes either at all relevant loci or at all but one relevant locus. Individuals with two or more heterozygous genotypes have more than one possible haplotype configuration compatible with their genotype data, and hence are ambiguous with respect to phase or haplotype information.

For example, consider two biallelic loci, one with alleles A and a and one with alleles B and b. An individual with a two-locus genotype (AA) and (BB) must have received two ‘A-B’ haplotypes. An individual with an (Aa) and a (BB) genotype must have received haplotypes ‘A-B’ and ‘a-B’ from his/her parents. The haplotypes of these two individuals are “unambiguous” for these two loci (i.e., their phase information for these loci is known). An individual with the genotypes (Aa) and (Bb) however, could have received haplotypes ‘A-B ’ and ‘a-b’, or haplotypes ‘A-b’ and ‘a-B’, and hence is ambiguous with respect phase information for these loci.

Thus, in general, in the absence of explicit phase information, if an individual is heterozygous at more than one locus, he or she is ambiguous with respect to haplotype and phase information. Individuals that are ambiguous thus have ‘missing’ or ‘incomplete’ phase information. Individuals that are unambiguous with respect to phase and haplotype data have ‘complete’ information.

Estimation of quantities, such as haplotype frequencies, from data in which only some individuals in the sample have complete information can be accomplished through statistical algorithms such as the E-M algorithm (Excoffier et al, Molecular Biology and Evolution, 12, 921-927 (1995); Hawley et al, Journal of Heredity, 86,409411 (1995); Long et al, American Journal of Human Genetics, 56, 799-810 (1995). The E-M algorithm and related algorithms use haplotype frequencies from unambiguous individuals to project and infer haplotypes for the ambiguous individuals.

The E-M algorithm first computes expected genotype probabilities based on haplotype frequency estimates provided by genotype data from individuals with complete information and projected frequency information for individuals that have ambiguous genotypes. This is the ‘expectation’ step. Once estimates of the frequencies are obtained, the probability of each possible pair of haplotypes for each individual's genotype configuration is computed. These probabilities provide information about how compatible the estimated haplotype frequencies are with the genotype data. This step is the ‘maximization’ step. These two steps are pursued in sequence until the estimates converge (i.e., do not change with subsequent expectation and maximization calculations).

E-M algorithm implementations for haplotype frequency estimation should be fast, given that the algorithm may require several iterations to converge. They also should be efficient in terms of information storage, since with many loci being evaluated there may be a large number of possible haplotype configurations for individuals with ambiguous genotype information. In addition, if tests are to be conducted on the estimated haplotype frequencies, the frequencies may have to be re-estimated many times, which could be very time consuming.

The method and software described herein for estimating haplotypes has many differences in computational efficiency and programming options compared with the Excoffier & Slatkin Arlequin software method (Excoffier et al. Microbiology & Evolution 12, 921-927 (1995)). In one embodiment, the system is optimized for use with SNP data, which only encompass 2-allele systems. In contrast, the Arlequin program allows for more than two alleles per locus for use with microsatellite data. Because the program/method in embodiments of the invention are written for a two allele system, calculating all haplotype and diplotype probabilities once and then retaining the values is feasible, and is computationally more efficient, and thus faster than a system where all possibilities have to be recalculated at every iteration. In contrast, it would be computationally prohibitive to retain all possible haplotypes and diplotype configurations (possible 2-haplotype combinations for individuals) across iterations for a program designed to handle more than two alleles. This means that the algorithm must recalculate these possibilities at every iteration, thus slowing the computational time dramatically. When several thousand analyses are necessary, which is likely to be the case, for example, when doing association studies for clinical trials, this is a considerable advantage.

Embodiments of the invention also differ from the Excoffier/Slatkin program in how they approach initial haplotype frequency values. Although E-M likelihood maximization algorithms have the desirable property that they will always approach a maximum, rather than a minimum value, this convergence may be slow, and may plateau at a ‘local’ maximum rather than the true, or ‘global’, maximum likelihood. This tendency to rest on local maxima means that these programs are sensitive to the initial values used to initiate the iterative process. For this reason, embodiments of the invention are designed to repeat the maximization process using several different starting points for as many random starting points as the user wishes, and then to survey over all of the maximum values to increase the confidence that a true global maximum is reached.

An additional version of the program was written in the “C” programming language and is significantly faster than the Fortran version due to several important factors.

1) Coding of the Haplotypes:

The E-M algorithm program described herein is based on haplotype frequency estimations. For SNPs, there are only two possibilities for a given haplotype for a given locus, either the frequent or the rare allele. Accordingly, embodiments of the present invention identify haplotypes using a binary (e.g., two state) code. A convention is set such that all possible haplotypes are coded with binary mask arrays. For example, for a given loci A/T, the haplotypes is 0 if the base is A and is 1 if the base is T. More generally, for each possible site, the first base in alphabetical order is 0 and the other base is 1. With this convention, all of the haplotypes can be coded with binary mask arrays. For example, if there are 5 SNPs: A/T C/G C/T A/G C/G, the haplotype ACTGC will be coded 00110.

There are two main advantages to this way of coding:

1) All operations on haplotypes become faster because binary operations are the most efficient ones due to the internal structure of the computer. Thus, efficient processes to generate/manipulate those haplotypes can be implemented.

2) In the algorithm and in its implementation in the program, you need to create some arrays that will store information about haplotypes. Those-arrays are composed of cells. It is important to keep track of which cell contains information about which haplotype. With a binary implementation, this problem no longer exists because for computers, binary mask arrays and integers are the same (more precisely, integers are stored in memory with binary numbers). So cells in arrays can be directly accessed with the haplotype itself.

For example, the haplotype ACTGC is coded 00110, which corresponds to 6 in decimal integer form. If information about its frequency is stored in the 6th cell of the array containing all frequencies, then there is a direct relation between the haplotype and its frequency. There is no need to keep track of which cell contains which information. This becomes implicit, thus increasing the efficiency of the program. This way of coding is particularly powerful for long haplotypes.

II) Grouping the Genotypes:

A number of operations involve a sum of various operations for each genotype. If genotypes of the same type are grouped, and assigned a factor equal to the number of people carrying each genotype, then one can avoid performing exactly the same operation several times. Instead, one can perform the operation one time and multiply the result by the number of people, thus obtaining the same result with fewer operations. The speed of the program is more enhanced when a small amount of sites are used because a few groups are generated with a lot of subject data in them.

For example, if there is a study of two loci with 200 subjects, there are only four possible haplotypes. Instead of performing an operation 200 times, and summing to obtain the desired results, embodiments of the program perform the operation four times, multiply the results by the number of subjects carrying the genotype, and then sum the totals for each individual carrying the genotype. Thus, the operation is performed only four times instead of 200.

II. Computing Test Statistics

The second part of the process for estimating haplotype frequencies is computing a test statistic that assesses evidence for estimated haplotype frequency differences between the cases and the controls (or any two groups). Relevant test statistics should preferably assess the association between the case and control haplotypes and the targeted disease, for example. At least two phenomena are relevant for constructing appropriate test statistics: 1) the test statistics should be able to identify individual haplotypes that differ in frequency between the cases and controls because they harbor disease-predisposing mutations; and 2) the test statistics should be able to identify subtle differences between overall haplotype frequency profiles between the case and controls.

Thus, two types of test statistics are used:

Individual haplotype test statistics. These test statistics are used to assess whether a particular haplotype is more frequent among the cases than the controls. These statistics should also indicate the overall contribution of the haplotype to disease prevalence, for example. This distinction is important since a particular haplotype can be more frequent among cases than controls, and still not be related to disease prevalence.

Overall or “Omnibus” test statistic. These statistics are used to assess the overall differences in haplotype frequency profiles between cases and controls. These statistics do not focus on a single haplotype, but rather consider all the haplotypes as a group or profile. This permits the discovery of multiple haplotypes that are greater in frequency (although possibly in more subtle ways) in cases rather than controls.

Initially, the null hypothesis for omnibus tests is that there is no difference in haplotype frequency profiles between the groups, regardless of the linkage disequilibrium between loci within any single group. A test to accomplish this is the ‘omnibus’ likelihood ratio test. The omnibus test compares the final likelihood of the estimated haplotype frequencies from an E-M procedure run on all groups combined (the null hypothesis that all groups come from the same distribution of haplotypes) versus the sum of the final likelihoods when haplotypes are estimated within each group is run through the E-M procedure separately. If this difference is significant, it can be inferred that the two or more groups have different haplotype frequency distributions.

To assess the statistical significance of this difference between haplotype frequencies, a permutation test is performed that simulates hypothetical data sets assuming the null hypothesis by ‘permuting’ the haplotypes among the cases and controls randomly. Specifically, data sets are simulated by randomly re-assigning one relevant item (the haplotype, for example) collected on the individuals in a sample and re-computing test statistics with the resulting ‘fake’ data sets. Test statistics resulting from these fake data sets are used to estimate a distribution for the test statistic. In the context of haplotype frequency differences tests with cases and controls, case and control status is reassigned randomly and the haplotype frequencies are re-estimated for comparison.

An alternative permutation is derived from “algorithm S” described in “The Art of Computer Programming” (Vol 2, pg 142). Briefly, each individual in the combined population is assigned a random number between 0 and the number of individuals yet to be assigned to a sub-population (1 or 2). If the random number is less than the number of individuals to be assigned to sub-population 1, or if there are no more individuals to be assigned to sub-population 2, then the individual is assigned to sub-population 1 and the number of individuals to be assigned to sub-population 1 is decreased by 1. Otherwise, assign the individual to sub-population 2 and decrease the number of individuals to be assigned to sub-population 2 by 1.

Accordingly, for each permuted set, the likelihood ratio test statistic is computed and compared with the value observed for the actual data set. The number of times a simulated data set statistic exceeds the observed value divided by the total number of simulations performed gives the probability of getting the observed statistic value by chance, and is thus an ‘empirical’ p value which can be used to make inferences.

The omnibus test described above detects several kinds of differences between haplotype frequencies among the groups, including single disease-association haplotypes, or varying combinations of disease-association haplotypes. In addition to this test, all possible single-haplotype chi-square tests can be calculated using a permutation-derived significance assessment. This method can provide two measures of association between groups for a particular haplotype, the Odds Ratio (OR) and the P-excess value.

The OR is equal to: (HF_{case}*(1−HF_{ccontrol}))/(HF_{control}*(1−HF_{case}))

with HF_{case}=haplotype frequency estimated for cases, and

with HF_{control}=haplotype frequency estimated for controls.

III. Drawing Inferences

Once test statistics have been calculated to determine the frequencies of haplotypes among cases and controls, their statistical significance is preferably assessed. The statistical significance of a test value is based on the probability that the test value could have resulted purely by chance. Thus, the determination is whether a test statistic value is so large (or small) that it is not likely to have occurred purely by chance. If the value did not occur by chance, the statistic is likely to have captured some true underlying relationship between the haplotypes and the target disease, for example. This statistical significance can then lead to inferences about the relationship between the haplotypes and the disease.

There are several methods that can be used to assess the probability that a test has occurred purely by chance. The issues that are addressed when using these methods include: 1) the error that arises because the haplotype frequencies were estimated rather than counted or observed directly; 2) the statistical difficulties associated with the presence of rare haplotypes either among the cases or controls or both, since the haplotypes may be rare due to poor estimation or for some biological reason (e.g., individuals possessing them are not viable); and 3) potential bias resulting from testing haplotype frequency differences in small sample sizes.

Methods for assessing the probability of observing a specific test statistic value purely by chance involve deriving the distribution of the test statistic and include:

Asymptotic Tests. Asymptotic theory relates to the behavior of statistical quantities such as test statistics as sample sizes approach infinity. For many statistical problems, such theory can be worked out analytically and can provide relevant methods for determining probabilities one can use for making inferences. Unfortunately, for estimated haplotype frequency based test statistics, the relevant mathematics are difficult. In addition, asymptotic results may not apply to finite (i.e., realistic) samples of certain sizes, and it is difficult to know what sample size is needed before one can reliably use asymptotic results.

Inference Based on Exact Distributions. One can attempt to enumerate all possible situations that could have arisen in a certain study and then merely calculate explicitly how often some observed test statistics, e.g., haplotype frequency difference, is likely to occur. Unfortunately, such enumeration is very difficult for tests involving estimated haplotype frequencies, since the number of possible situations is astronomical.

Inference Based on Empirical Distributions. One way of assessing how often a certain event (e.g., haplotype frequency differences) is likely to occur is to compile data on frequencies of similar events and then use this compiled data to draw inferences about the event in question.

Parametric Bootstrap Tests. To derive a probability for a certain event, one needs to consider the probability distribution of outcomes that include the event in question. Although such distributions can be derived analytically in certain instances (via asymptotic theory) they are difficult to derive and are often assumption-laden. As an alternative, one can simulate events and estimate a distribution from these simulated events. This can be done in several ways. A ‘working’ distribution for the observations (e.g., haplotype frequencies) can be assumed rather than the test statistic based on what was observed (e.g., haplotype frequency differences between cases and controls) and then generate hypothetical observations from this distribution. Test statistics computed from these simulated observations can then be used to estimate a distribution which can, in turn, be used to assess the probability of observing the actual (i e., real data) outcome. One may be able to derive the working distribution for an event analytically, but this is often difficult. As an alternative, one can use simulations, as described. The use of such simulations provides a “Monte Carlo” approximation to the bootstrap distribution.

Non-parametric Bootstrap. As an alternative to generating simulated observations from a distribution, observations can be resampled from actual data to generate ‘fake’ data sets that are then subjected to an analysis (e.g., haplotype frequency difference analysis) and ultimately used to estimate a distribution. Since no distribution is assumed to generate the simulated observations, but rather actual data is resampled with replacement, this strategy is known as ‘non-parametric bootstrap’ re sampling.

Randomization Tests. As an alternative simulation-based test distribution estimation procedure to bootstrap methods, data sets can be simulated by merely randomly re-assigning one relevant item collected on the individuals in a sample and recomputing test statistics with the resulting ‘fake’ data sets. Test statistics resulting from these fake data sets can be used to estimate a distribution for the test statistic. In the context of haplotype frequency differences tests with cases and controls, case and control status can be reassigned randomly.

IV. SNP Haplotype Estimation Program

The haplotype estimation procedure of our program is related to the method outlined by Excoffier et al, Molecular Biology & Evolution 12, 921-927 (1995). The overall likelihood of the data can be expressed as the product of the probabilities of each observed ‘haplo-phenotype’ (set of phase unknown genotypes for an individual) multiplied by a multinomial constant. These haplo-phenotype probabilities can be expressed as the sum of the probabilities of all genotypic combinations possible for each particular haplo-phenotype, i, such that the final likelihood for the data is:

L(f₁,f₂, . . . fₕ)=constant*Π_{i=1, . . . m}[Σ_{g=1, . . . ci}P(h_{igk}h_{igl})]ⁿⁱ

where m denotes the number of different haplo-phenotypes observed in the data set; cᵢ denotes the count of all possible diplotypes for a particular haplo-phenotype i; h_{igk}/h_{igl} denote the two constituent haplotypes for a particular diplotype g; and nᵢ denotes the number of individuals with haplo-phenotype i.

Each iteration of the E-M algorithm obtains expected diplotype frequencies P(g=hₖhₗ)^{(j)} given the observed haplo-phenotypes and the haplotype frequency estimates at the previous iteration. This is calculated as the probability of the particular diplotype, g, among all possible diplotypes for phenotype i, weighted by the proportion of individuals with phenotype i:

P(g=hₖhₗ)⁽ⁱ⁾=Σᵢ{(nᵢ/n)[P(h_{gk}h_{gl})^{(j)}/Σ_{g=1, . . . ci}P(h_{igk}h_{ig1})⁽ⁱ⁾]},

where P(h_{gk}h_{gl})⁽ⁱ⁾ depends on the haplotype frequencies ((fₖ^{(j-1)})² if hₖ=hₗ; 2*fₖ^{(j-1)}*fₗ^{(j-1)} otherwise. These expected diplotype frequencies are then used to calculate new haplotype frequencies f₁^{(j)}, . . . ,fₕ^{(j)}, as fₜ^{(j)}=0.5 ΣᵢΣ_{g}δ_{gt}P(h_{igk}h_{igl})^{(j)} (where δ_{gt}=0 if hₜ not in diplotype g; l if hₜ occurs once in g; 2 if hₜ occurs twice in g). These frequencies are in turn used to calculate new expected P(hₖhₗ)^{(j+1)}, and so on until convergence is reached.

One advantage of embodiments of our program is the specific tailoring for diallelic loci, allowing all possible haplotypes (2^{L-#loci}) and diplotype configurations for each phenotype (2^{#heterozygous loci-1}) to be derived at the beginning of the process and stored for retrieval throughout the iterations and restarts. This reduces the amount of computational time as well as memory overhead needed to perform all the calculations.

One embodiment of the process begins with user-specified initial haplotype frequencies if desired, but by default chooses random values, constrained so that they sum to 1. To reduce the possibility of convergence to a local rather than the global maximum, the instructions will re-run the E-M algorithm on the same data using a new set of randomly chosen initial values. The number of ‘restarts’ can be specified by the user, as well as the convergence criterion and maximum iterations allowed per run.

Other advantages of the methods and procedures of the invention relating to a software program that embodies the language C are described in Section I.

V. Accuracy of Haplotype Frequency Estimation

The accuracy of methods for estimating haplotype frequencies was studied using a suite of computer programs designed to accommodate many computational problems thought to plague the use of the E-M algorithm (such as a potential for convergence to local maxima). The accuracy of haplotype frequency estimations via the E-M algorithm was also investigated as a function of a number of factors, including: 1) sample size, 2) number of loci studied, 3) haplotype and allele frequencies, and 4) locus specific allelic departures from Hardy-Weinberg and linkage equilibrium.

Previously, Excoffier et al, Molecular Biology & Evolution 12, 921-927 (1995). showed that their program's accuracy improved with large sample sizes, and was relatively insensitive to the recombination fraction. Using the methods described herein, we have found that the E-M algorithm produces accurate haplotype frequency estimates even for biallelic loci with alleles departing from Hardy-Weinberg equilibrium (HWE). In addition, many factors that may influence accuracy can be assessed empirically within a data set—a fact which can be used create ‘diagnostics’ that a user can turn to for assessing potential inaccuracies in estimation. The methods used to study the accuracy of haplotype frequency estimations are presented in Example 1.

Our method for haplotype frequency estimation from di-allelic diploid data performs very well under a wide range of population and data set scenarios. It is highly accurate even at extreme parameters. On average, for 5-locus haplotypes, the 60% estimates lie within a 0.03% interval and the 96% estimates lie within a 0.06% interval.

The improvement in larger samples is related to two factors. First, the algorithm assumes HWE, and larger sample sizes provide better representation of HWE. Second, the algorithm relies on multiple copies of the same haplotype in the data set, and larger samples provide a smaller ratio of haplotypes/total observations (i.e. more copies of the same haplotype).

As previously noted, there is relatively little influence of Hardy-Weinberg Disequilibrium (HWD) on accuracy, probably because of the little amount of missing data that contributes to estimates, especially when HWD is excess homozygosity. Although HWD may have small effect on estimation procedure, as you approach a disease gene, HWE will be lost (especially if recessive), so may have the greatest HWD at point of interest.

VI. Implementation of Methods of the Invention

The automated system for estimating haplotype frequencies can be implemented through a variety of combinations of computer hardware and software. In one implementation, the computer hardware is a high-speed multi-processor computer running a well-known operating system, such as UNIX. The computer should preferably be able to calculate millions, tens of millions, billions or more possible allelic variations per second. This amount of speed is advantageous for determining the statistical significance of the various distributions of haplotypes within a reasonable period of time. Such computers are manufactured by companies such as International Business Machines, Hitachi, DEC, and Cray. Currently available personal computers using single or multiple microprocessors should also function within the parameters of the present invention.

Preferably, the software that runs the calculations for the present invention is written in a language that is designed run within the UNIX operating system. The software language can be, for example, C, C++, Fortran, Perl, Pascal, Cobol or any other well-known computer language. It should be noted that the nucleic acid sequence data will be stored in a database and accessed by the software of the present invention. These programming languages are commercially available from a variety of companies such as Microsoft, Digital Equipment Corporation, and Borland International.

In addition, the software described herein can be stored on several different types of media. For example, the software can be stored on floppy disks, hard disks, CD-ROMs, Electrically Erasable Programmable Read Only Memory, Random Access Memory or any other type of programmed storage media.

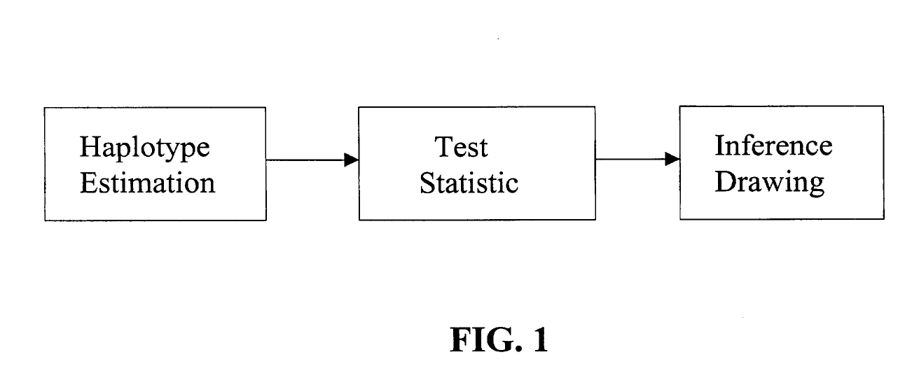

Referring to FIG. 1, a block diagram of an overall process 2 of drawing an inference is illustrated. The process 2 begins with a haplotype estimation 4 and then moves to calculation of a test statistic 6. The process 2 then finishes with drawing inferences 8 based on the haplotype estimation and the test statistic.

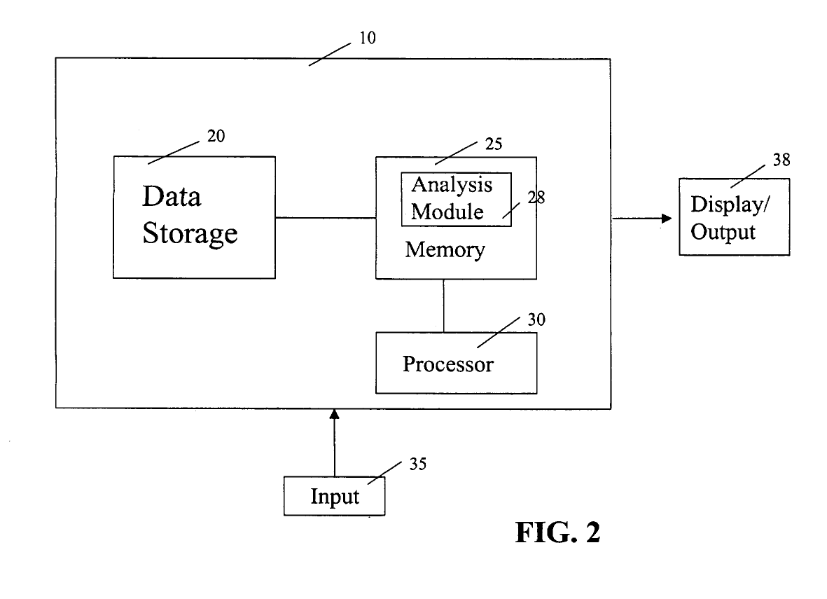

Referring now to FIG. 2, a system 10 that includes a data storage 20, such as that described above, is linked to a memory 25. Associated with the memory 25 is an analysis module 28 that stores commands and instructions for providing the data analysis described below. Communicating with the memory 25 is a processor 30 that is used to process the information being analyzed within the analysis module 28. Conventional processors, such as those made by Intel, Digital Equipment Corporation and Motorola are anticipated to function within the scope of the present invention. As illustrated, an input 35 provides data to the system 10. The input 35 can be a keyboard, mouse, data link, or any other mechanism known in the art for providing data to a computer system. In addition, a display 38 is provided to display the output of the analysis undertaken by the analysis module 28.

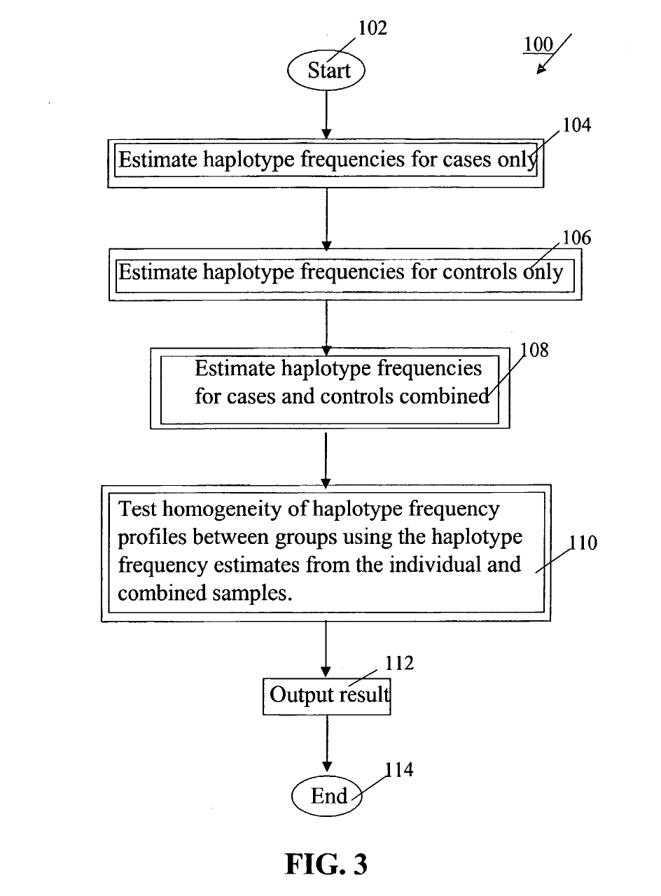

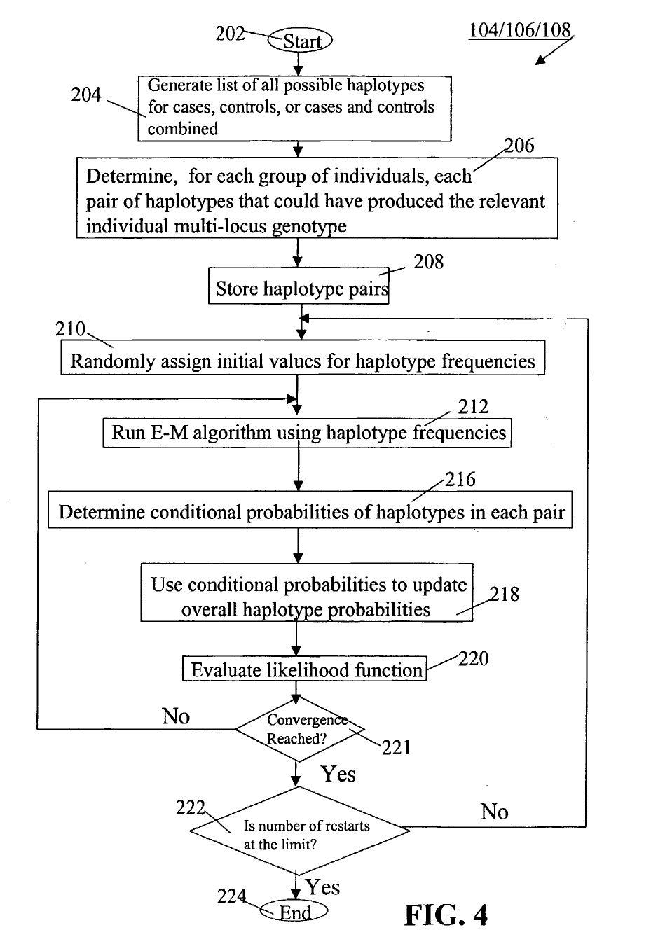

Referring now to FIG. 3, a process 100 of estimating haplotype frequencies from DNA marker genetic data is illustrated. The process 100 begins at a start state 102 and then moves to a process state 104 wherein an estimate of haplotype frequencies for cases only is determined. The process 104 is described in more detail with regard to FIG. 4. The process 100 then moves to a process state 106 wherein an estimate for the haplotype frequencies for controls only is determined. The process 100 then moves to a process state 108 wherein an estimate for the haplotype frequencies for cases and controls combined is determined. This process is illustrated in more detail with reference to FIG. 4 below. It should be realized, of course, that the process states 104, 106 and 108 can be performed in any order within the present system.

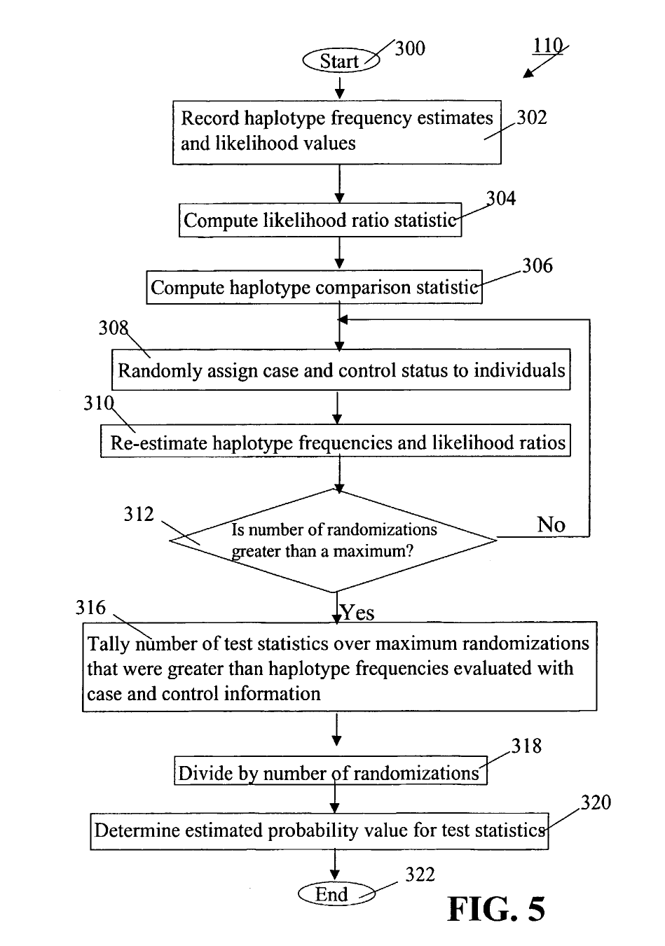

Once an estimate of the haplotype frequencies for cases, controls, and cases/controls combined has been generated, the process 100 moves to a process state 110 wherein the homogeneity of haplotype frequency profiles between the various groups is tested based on the haplotype frequency estimates generated in process states 104, 106 and 108. The process 110 is described more completely with regard to FIG. 5. The process 100 then moves to a state 112 wherein the result is output to a display or printer. The process 100 then terminates at an end state 114.

Referring now to FIG. 4, a process 200 for estimating haplotype frequencies of cases, controls, or combined cases/controls is illustrated. The process 200 begins at a start state 202 and then moves to a state 204 wherein a list of all possible haplotypes is generated. The process 200 then moves to a state 206 wherein, for each group of individuals, each pair of haplotypes that could have produced the relevant individual multilocus genotype is determined.

The process 200 then moves to a state 208 wherein the haplotype pairs are stored to a memory within the system 10. The process 200 then moves to a state 210 wherein the initial values for the haplotype frequencies are randomly assigned. The use of the E-M algorithm is described hereafter.

The process 200 then moves to a state 216 where the estimation step of the E-M algorithm to determine the conditional probabilities of haplotypes within each pair of haplotypes is conducted. The process 200 then moves to a state 218 that corresponds to the maximization step of the E-M algorithm wherein the conditional probabilities are used to update the overall haplotype probabilities.

The process 200 then moves to a state 220 wherein a likelihood function of the haplotype probabilities is evaluated. A determination is then made at a decision state 221 whether convergence of the likelihood functions has taken place. If convergence has not taken place, the process 200 returns to the state 216 to run the expectation step of the expectation-maximization algorithm again.

However, if a determination is made at the decision state 220 that convergence has taken place, the E-M algorithm is finished. The process 200 then moves to a decision state 222 to determine whether the number of restarts has reached a maximum limit. If the number of restarts is at a limit, the process 200 terminates at an end state 224. However, if the number of restarts has not reached a limit, the process 200 returns to the state 210 to randomly assign initial values for the various haplotype frequencies.

Referring now to FIG. 5, the process 110 of testing homogeneity of haplotype frequency profiles between groups is illustrated. The process 110 begins at a start state 300 and then moves to a state 302 to record haplotype frequency estimates and likelihood values.

The process 110 then moves to a state 304 wherein the likelihood ratio statistic is computed. The process 110 then moves to a state 306 wherein the haplotype comparison statistic is computed.

The process 110 then moves to a state 308 wherein the case and control status is randomly assigned to various individuals in the group. Once the status has been randomly assigned, the process 110 then moves to a state 310 wherein the haplotype frequencies and likelihood ratios are re-estimated based on the randomly assigned case and control status'. A determination is then made at the decision state 312 whether the number of randomization's is greater then a maximum value. If a determination is made that the number of randomizations are not greater than the maximum, the process 110 returns to the state 308 wherein the case and control status is randomly re-assigned to various individuals.

However, if a determination is made at the decision state 312 that the number of randomizations is greater then a maximum, the process 110 then moves to a state 316 wherein the number of test statistics that were greater than the observed statistic for the true case and control groupings is tallied over the randomizations.

The process 110 then moves to a state 318 wherein the number of test statistics tallied at the state 316 is divided by the number of randomizations. A determination is then made at a state 320 of the estimated probability value for the test statistics based on the number of randomizations. The process 110 then terminates at an end state 322.

### EXAMPLES

The following examples are provided to further describe the invention, not as a means of limitation.

### Example 1

### Tests of the Accuracy of Haplotype Estimation

To test the accuracy of haplotype estimation using the methods described above, the error between E-M-based haplotype frequency estimates and either haplotype frequencies observed in particular data sets or the true haplotype frequencies in the population at large, was assessed as a function of several population and data set characteristics. The possible factors influencing the accuracy of the method include sample size (and sampling error), proportion of ambiguous individuals/heterozygous loci, presence of HWE, haplotype and allele frequencies, number of loci in haplotype, and level of linkage disequilibrium in the area.

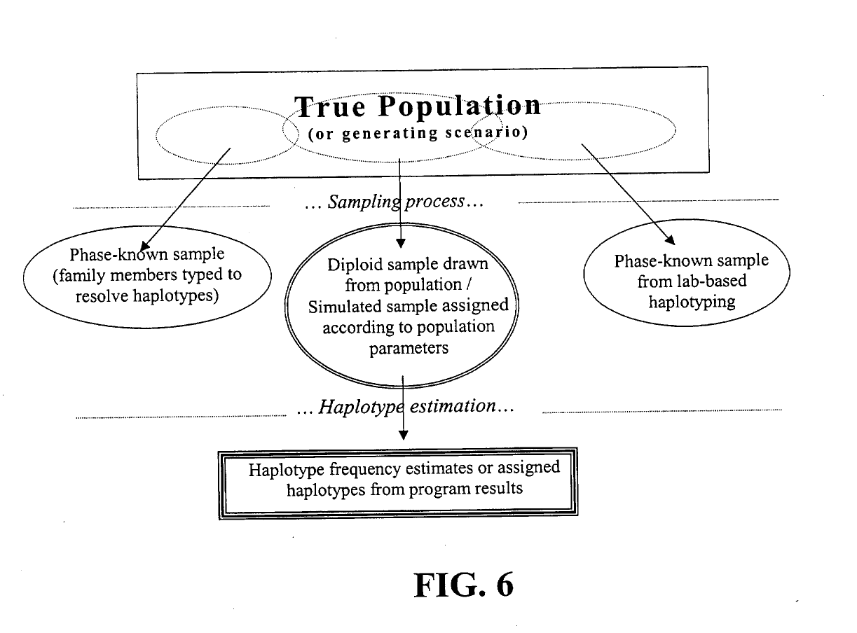

Sample diploid data sets were simulated using computer programs that perform one embodiment of our method under different generating (or true population) scenarios. The “accuracy” of our method was assessed by comparing the final estimated haplotype frequencies (E_{f}) to either the original generating frequencies (population parameters (G_{f})), or to the haplotype frequencies in a sample drawn from the simulation parameters (which are different than the generating frequencies due to sampling error/chance (S_{f})). The distinction between these comparison standards is illustrated in FIG. 6. If the main interest is assessing the overall validity of haplotype estimates representative of the true population parameters, the comparison of interest would be the estimated versus generating values. However, this comparison includes the effect of sampling error, which would exist for the phase-known methods. A more relevant comparison for practical purposes, then, would be the accuracy of a haplotype estimation from a sample diploid set (simulated from the generating parameters), as this more closely reflects any additional error incurred by our estimation procedures relative to phase-known methods from population samples.

Simulation

Data sets of varying sample sizes were simulated by randomly assigning haplotypes with a specific number of di-allelic loci to all individuals. Haplotype frequencies were either constrained to be equally frequent (each=½^{L}) among the n individuals, or were generated according to a specified variance parameter indicating amount of departure from uniformly distributed frequencies. For example, a simulation with haplotype frequency variance set at 10, would generate and randomly assign haplotypes among the n individuals according to a distribution with mean ½^{L} and variance 10, resulting in very large discrepancies between haplotype frequencies within the data set. Simulation in this way is not based on a particular population genetics model, but samples over many underlying allele and haplotype frequencies, allelic association strengths, and Hardy-Weinberg scenarios, allowing for the assessment of the influence of such characteristics on estimation validity.

Measures of Estimation Accuracy

The choice of accuracy measurement depends on the study goals. In our case, the most interesting result is the accurate estimation of haplotype frequencies, rather than the identification of any particular haplotype. Thus, we have used two measures of accuracy for frequency comparison—absolute difference (or bias) between the estimated frequency of any randomly chosen haplotype and its frequency in the comparison sample or population, and the mean squared error between all haplotypes of the two comparison groups.

In a 2-locus system, the absolute difference between the generating, sample, and estimated haplotype frequencies, could be calculated for all four possible haplotypes. For example, the |bias| between the frequency of haplotype 1, h₁, from the generating parameters and the final estimated frequency would be |G₁−E₁|. However, as the number of loci increase, recording this value for every possible haplotype and every possible comparison would be prohibitive. Instead, the absolute bias is calculated for the most and least frequent haplotypes, as well as for a random estimated haplotype from each simulated data set. In order to incorporate differences among all haplotypes, and to standardize for the number of possible haplotypes in a data set, the mean standard error between the three stages (generating, simulated sample, and estimated haplotypes) is also calculated. For example, the mean standard error (MSE) of estimates compared to generating values would be MSE_{g-e}=Σₕ(Eₕ−Gₕ)²/Nₕ for h=1 . . . 2^{L}.

Results

To set optimal conditions for measuring the effect of population and data set characteristics on the haplotype estimation accuracy, the influence of several program specifications were assessed, including restarts, number of iterations, and the size of the convergence criterion. It was found that because the E-M algorithm may converge slowly and to a local maximum, the program should be restarted several times, with different initial values, ample iterations, and a small enough convergence criterion to achieve the global maximum. Varying these three programming options can guide the most efficient maximization.

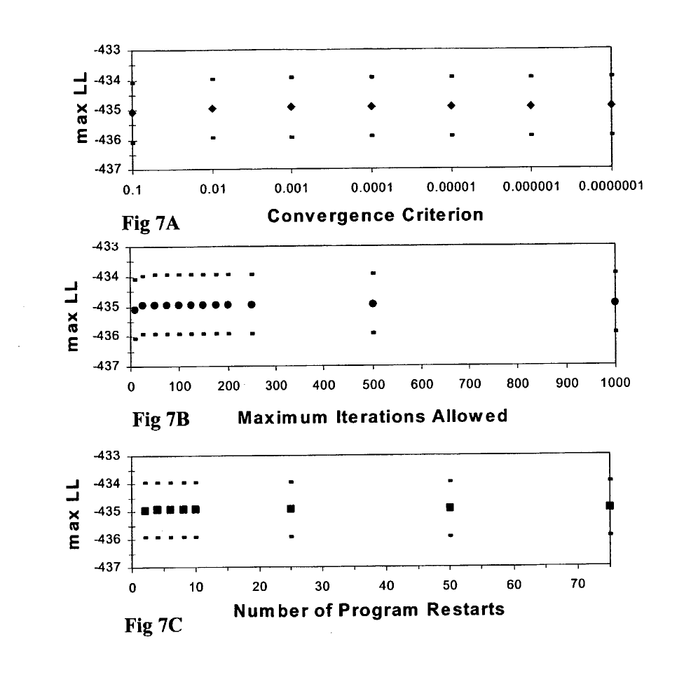

The distribution of resulting log-likelihoods and error measurements also provides an indication of the correctness of the maximization process. FIG. 7 shows the expected increase and plateau of log-likelihoods as the three options become increasingly liberal for runs performed on the same batch of 500 simulated data sets. From these results, setting the program for 10 restarts, setting maxit=150, and convergence to 0.00001, should be reasonably efficient. These settings were then used for all subsequent program runs described in the following sections.

There is no apparent trend in mean squared error or bias between the estimates and sample/generating values as a function of these settings, although the standard deviation of the averaged maximum log-likelihoods decreases as the settings become more liberal. In this situation, each batch is a new set of simulated data sets, as opposed to the results shown in FIG. 7 in which all runs were performed on the same batch of 500 simulated sets such that likelihoods were comparable.

Population Issues

Sampling Error

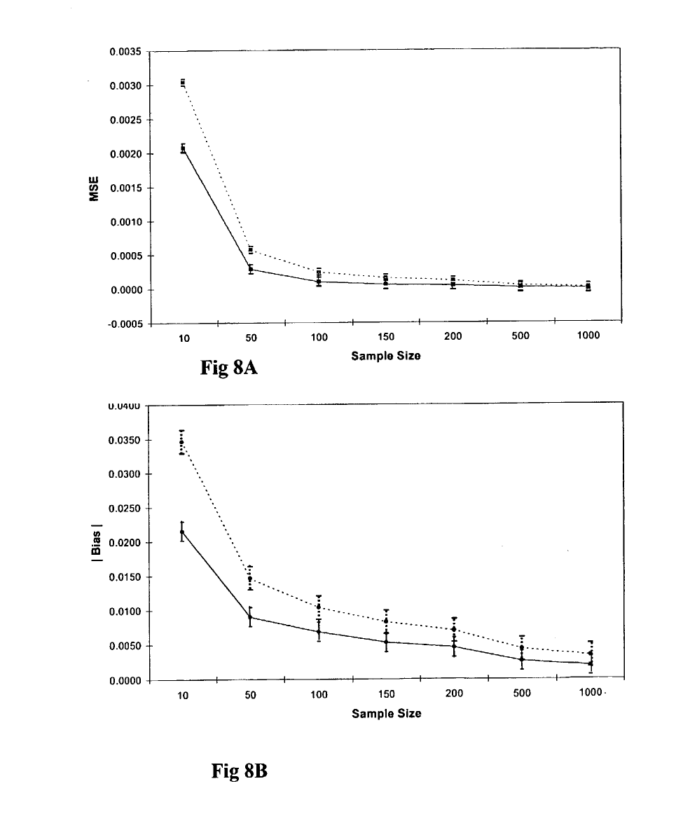

Much of the error between the true (generating) population parameters and those estimated from the sample is due to sampling error itself, rather than to error from the estimation procedure. This error due to sampling alone can be seen by the decrease in absolute bias and mean squared error as the sampling size increases (FIG. 8). This can also be seen in the relative amount of overall MSE from the generating to estimated values that is accounted for in the generating-to-sample error (see Table 1).

Missing Data

The amount of missing data (e.g., ambiguous genotype data) in a particular sample will influence the validity of the estimates, due to the algorithm's weighting towards observed unambiguous data. The amount of missing data could be assessed within the sample as the proportion of ambiguous individuals (more than two possible haplotypes can explain the observed multi-locus genotype, i.e., >1 heterozygous locus), or this could be represented more crudely by the number of homozygous loci in the data set.

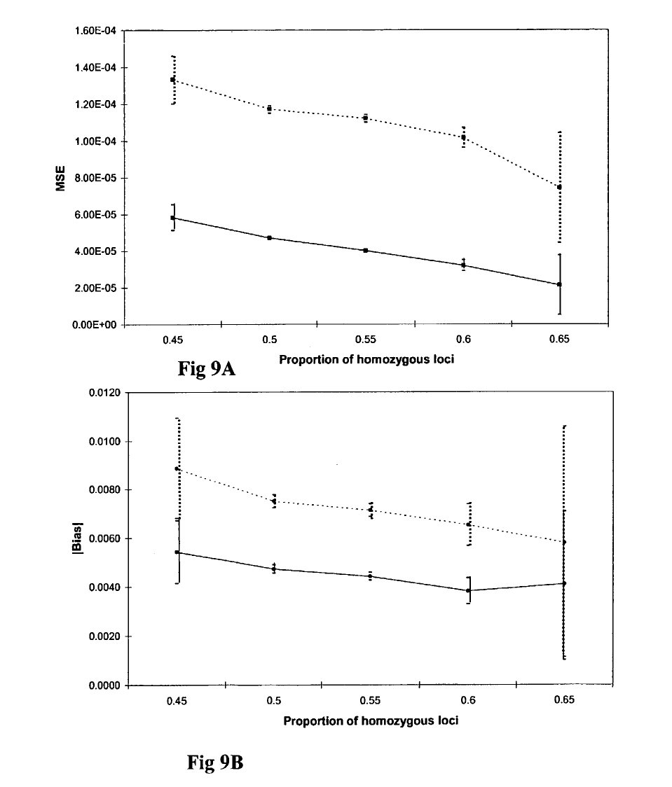

FIG. 9 is a line graph illustrating the effect of the proportion of homozygous loci in the data set on the accuracy of the measure. As would be expected, there is a substantial loss of accuracy as the amount of missing data increases. However, even at the worst levels of missing data observed in our sets, the overall accuracy of the estimation is very good (max(bias)=0.01 difference between haplotype frequencies).

Differing Haplotype Frequencies

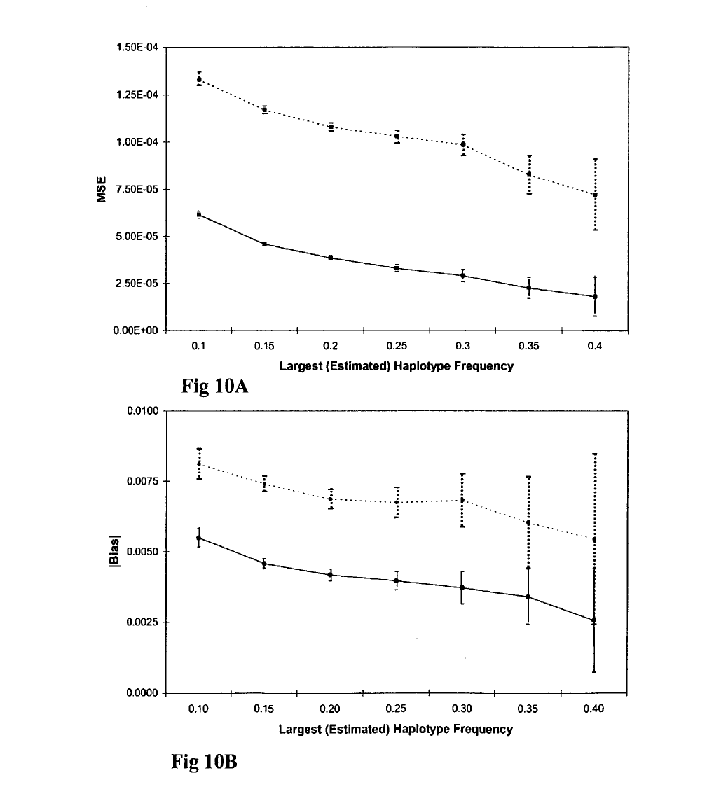

To test the accuracy of our program estimates under differing haplotype frequencies, both measures of accuracy were plotted by the highest haplotype frequency per data set, resulting in an increase in accuracy with increased haplotype frequency (FIG. 10). This would follow from the idea that estimates will be better when there are some very common haplotypes and thus many very rare haplotypes in the population. However, when the haplotypes are more equally frequent (as when LD does not exist between the loci), the estimation of frequencies is less accurate. To demonstrate this, accuracy was plotted by the simulated variance in haplotype frequencies. As the true haplotype frequencies become increasingly unequal, the accuracy of the program estimation increases (FIG. 10).

To demonstrate this another way, a chi-square test of homogeneity between the estimated haplotype frequencies (such that expected would be uniformity, or equal haplotype frequencies of ½^{L} for each haplotype) was performed, and the estimation accuracy was plotted by the chi-square “uniformity” value. Again, the accuracy of the program estimates increases as the haplotypes become more unequally distributed.

Allele Frequency

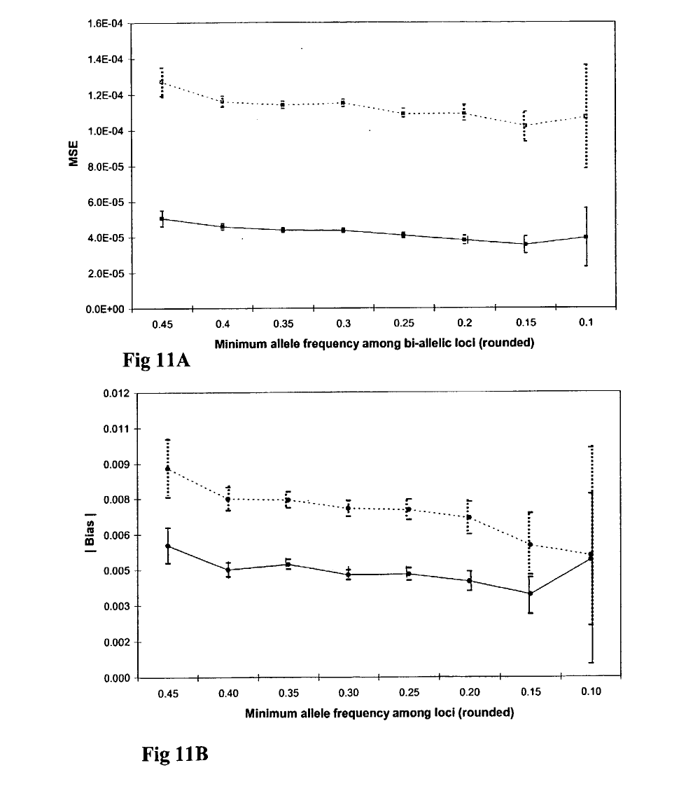

A factor somewhat related to haplotype frequency is the allele frequency in the population and sample. Following the results above, it may be expected that the more unequal the allele frequencies at each locus, the better the program's accuracy. This could be assessed in several ways, such as with a plotting program MSE and bias by the average smaller allele frequency across the loci, or plotting accuracy by the minimum allele frequency across loci. FIG. 11 shows the decrease in accuracy as the average smaller allele frequency approaches 0.5 (and thus, allele frequencies become more uniform).

Departures from Hardy Weinberg Equilibrium

Departures from Hardy-Weinberg may be a substantial source of error in E-M haplotype estimation because the algorithm relies on HWE in the expectation step. For this reason, one may expect to lose estimate accuracy when alleles at the constituent loci are not in HWE. However, departures from HWE may be due to excess homozygosity, which would simultaneously mean a decrease in the amount of missing data for haplotype resolution, so estimate accuracy may increase under such a scenario.

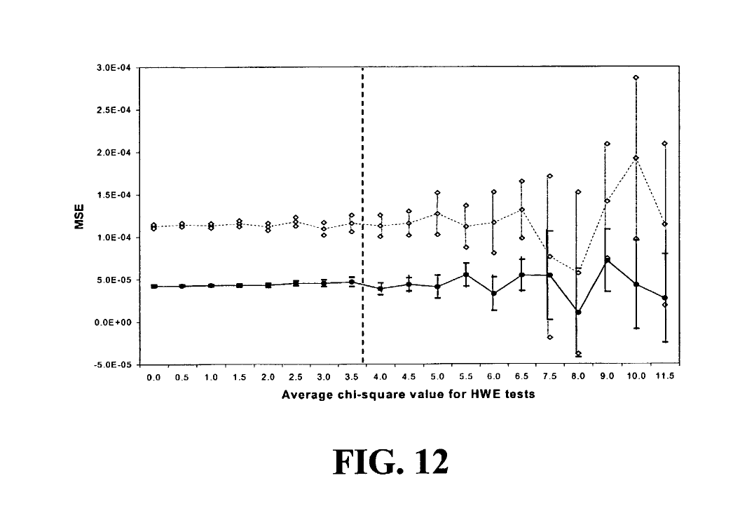

To assess this possible affect, and its direction on estimation accuracy empirically, the chi-square value of a HWE test at each locus in the sample population, and the direction of homozygosity (excess, or decreased amount of homozygotes compared to that expected under HWE) was calculated. The average HWE chi-square value across constituent loci, or the number of loci with HWE chi-square values above 3.84, by our accuracy measures, was plotted. These show very little effect of HW disequilibrium on the haplotype estimation accuracy, although the variance in error among data sets increases with departure from HWE (FIG. 12). The data sets were separated into three groups: those with an average HWE chi-square value <3.84, and those with significant H-W disequilibrium separated into excess homozygosity and excess heterozygosity. The average MSE between haplotype frequency estimates and sample data sets was greater for the excess heterozygote group, versus the other two, although this trend was not statistically significant.

Amount of Linkage Disequilibrium

The amount of linkage disequilibrium between the constituent loci preferably has an important effect on the haplotype estimation, because haplotypes will be inconsistent among loci in complete equilibrium. There are several choices in measuring the amount of LD in the area, including pairwise D′ values or the associated χ² values for a test of equilibrium. From these, the entire matrix of pairwise values, or only the neighboring locus pairwise values can be averaged. For validity measures as a function of the average chi-square value of the pairwise LD matrix, the error levels appear to be consistent across the significant LD values, and show slightly more variance when the average LD is not significant. Plots of the other measures of LD mentioned show similar results.

Number of Loci

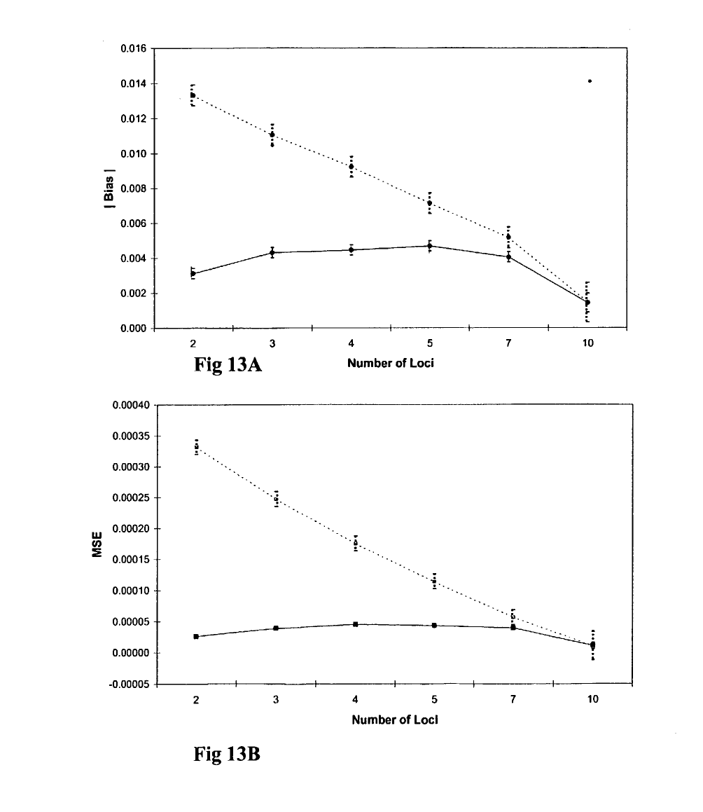

The reliability of haplotype frequency estimation for different numbers of loci is also important. FIG. 13 shows the overall increase in accuracy as the number of constituent loci increases. However, this figure also shows the u-shaped distribution of error and bias between the sample and estimated haplotype frequencies. This is due mostly to the decrease in error between the generating simulation values and the sample data set as the number of loci increase. The decrease in error probably reflects the decreasing orders of magnitude in the haplotype frequencies themselves as the number of loci increases. The distribution between the sample and estimates is likely more of interest here. The u-shaped distribution may reflect the initial decrease in accuracy as the number of constituent loci increases, as may be intuitive. The later ascent in accuracy may be due to the greatly decreased absolute difference between haplotype frequencies with such a high number of loci.

Discussion

Haplotype frequency estimation for di-allelic diploid genotype samples performs very well under a wide range of generating-population and sample-specific situations. In fact, even the worst haplotype frequency estimates were accurate (for 5-locus haplotypes, 60% of the estimates lie within 3% of their generating values and 96% lie within 6% of their generating values). The majority of overall error between the original population parameters and the final frequency estimates is due to sampling error, rather than to algorithmic and estimation problems or inaccuracies. This is supported by the increase in overall accuracy with increasing sample size. This improvement with sample size is likely a function of several factors: 1) the E-M algorithm assumes HWE, and larger sample sizes provide better representation of HWE if it truly exists in the source population; and 2) the algorithm works best with low amounts of “ambiguous” individuals (i.e., individuals with unresolvable phase information) and larger sample sizes also provide a greater number of unambiguous individuals.

Estimation accuracy tends to be dependent on the uniformity of allele and haplotype frequencies. As the haplotype frequencies become more unequal, the more frequent haplotypes can be estimated accurately and a large number of 0.0 frequency (i.e., rare) haplotypes will lead to accurate estimates of many of them, since they won't exist in the sample and be estimated as 0.0 frequency haplotypes. Thus, when many haplotypes have zero frequency, their absence in the data set will generally allow accurate estimation of this zero frequency, contributing to a small overall error in frequency estimation.

The relatively weak influence of departures from HWE on estimation accuracy is of extreme interest. It might be expected that departures from HWE, given the E-M algorithm's exploitation of HWE to compute expected haplotype frequencies, would significantly influence accuracy of the resulting estimates. However, as noted recently by Osier et al. American Journal of Human Genetics 64, 1147-1157(1999) there is balance between loss of accuracy due to departure from HWE and gain of accuracy due to the decrease in missing phase information with an excess of homozygosity, as might result from departures of HWE. This example illustrates this as well, as departures from HWE that result in an excess heterozygosity do lose accuracy while those that result in an excess homozygosity do not. This issue is of particular relevance when one has sampled diseased individuals, since an excess homozygosity at the disease allele (and all alleles at loci in LD with it), may be expected, especially if the disease is recessive (Nielsen et al, American Journal of Human Genetics 5, 1531-1540 (1998)).

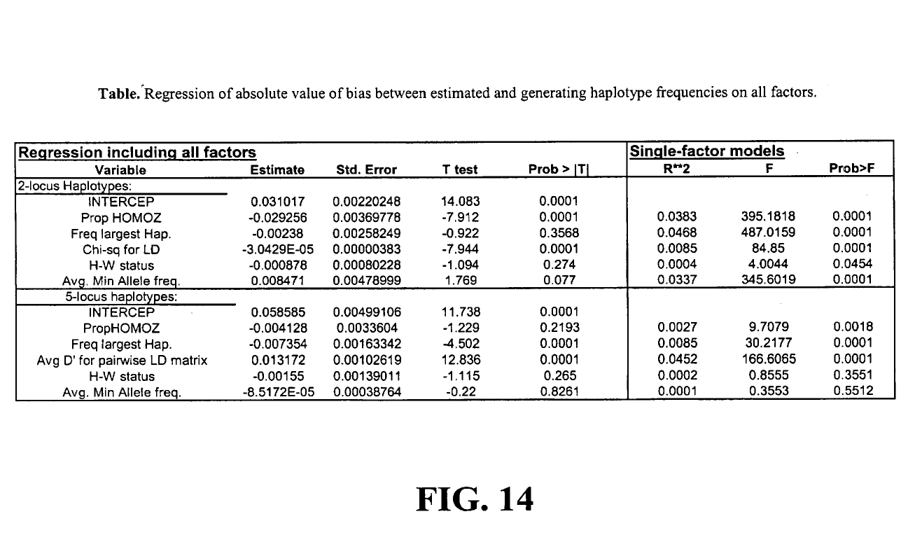

Use of a regression model to assess the simultaneous effect of different factors on estimated accuracy also has some utility. Many of the factors can be assessed within a given data set (e.g., evidence for departure from HWE, number of heterozygous genotypes, number of individuals with two or more heterozygous genotypes, etc.). Thus, one can predict MSE or bias through the regression model outcomes, with their own data. The results of this prediction can then serve as a “diagnostic” for potential inaccuracies in haplotype frequency estimates due to features in the relevant data set (FIG. 14).

Ultimately, the results of our studies suggest that even in the worst cases, individual haplotype frequency estimates via the E-M algorithm don't deviate much beyond 5% of their true value for sample sizes of 100 or greater. Finally, the results refer to the accuracy of haplotype frequency estimation only. The extent to which the factors we studied influence any statistical inference procedures that make use of haplotype frequency estimates demands independent attention.

### Example 2

Case/Control Haplotype Analysis

This example describes methods for testing associations between estimated haplotype frequencies derived from multilocus genotype data and disease endpoints assuming a simple case/control sampling design. These methods overcome the lack of phase information usually associated with samples of unrelated individuals and provide a comprehensive way of assessing the relationship of a sequence or multiple-site variation and traits and diseases within populations. The study is of the relationship between polymorphisms within the APOE gene locus and Alzheimer's disease. The results confirm the known association between the APOE locus and Alzheimer's disease, even when the polymorphism is not contained in tested haplotypes. Thus, linkage disequilibrium-induced associations between polymorphisms that neighbor a functional polymorphism and a disease may be detected in large, freely-mixing populations using estimated haplotype frequency methods.

The 223 AD cases and 159 non-demented elderly controls were sampled from greater France and are likely to be characteristic of the type of heterogeneous samples one might expect to obtain from large, freely-mixing populations. The total size of the region encompassing the eight SNPs studied within the APOE gene region was approximately 200 kb. Another set of five SNPs in a region on chromosome 13 were also analyzed as a control.

Methods

Sampling & Genotyping.

Alzheimer's patients were sampled from hospitals in France. Controls were also obtained from greater France. On enrollment in the study, a blood sample was obtained, DNA was extracted, and genotyping was performed as described previously in patent application Ser. No. 09/438,016 (hereby incorporated by reference herein in its entirety including any drawings, figures, or tables). The average age of the Alzheimer's patients was 73.4 (±10.0 standard deviations) and the average age of the controls was 71.3 (±5.0). This difference was significant (p=0.017) by student's t-test.

Pairwise Locus Disequilibrium Analysis.

Alleles at pairs of loci were assessed for linkage disequilibrium (LD) using the composite test described by Weir. The measure of LD known as D′ (Lewontin), which is corrected for allele frequencies at each of the loci was computed as well.

Haplotype Frequency Estimation.

Haplotype frequencies were estimated via the method of maximum likelihood ⁸ from genotype data through the use of the Expectation-Maximization algorithm (⁹⁻¹¹). The accuracy of the E-M based estimates is quite good, even when some of the alleles at the loci are not in Hardy-Weinberg equilibrium, for moderate to large sample sizes (12-14).

Hypothesis Testing Procedures.

Single locus hypothesis tests were conducted by examining allele and genotype frequencies between the case and control groups using standard chi-square statistics for contingency tables ¹⁵. Two haplotype-based hypothesis tests were conducted. The first, an “Omnibus” likelihood ratio test, was pursued which examines the differences in haplotype frequency profiles between the case and control groups (as opposed to comparing particular haplotypes). A likelihood ratio statistic was computed from the estimated haplotype frequencies. This was pursued by computing a likelihood assuming equality of frequencies and then a likelihood allowing the frequencies to the unequaled forming the ratio of results. The null distribution of this LR statistic was then approximated via randomization tests in which case/control status indicators were randomly permuted among the individuals in the sample and likelihood ratio statistics recomputed ¹⁶.

The second haplotype-based hypothesis test focused on the differences in individual haplotype frequencies between the case and control groups. A chi-square statistic was derived from a simple 2×2 table based on the frequency of each haplotype versus all others combined in the case and control groups ¹⁵. The distribution of this test statistic (for each haplotype) was then approximated via permutation tests as well.

Results

Single-Locus Analyses.

Table 1 shows the results of single locus analyses with the 8 SNPs in the APOE gene region and 5 other SNPs on chromosome 13. Only two SNPs in the APOE gene region showed significant single locus associations with Alzheimer's disease. The SNPs with the strongest association were a SNP responsible for the e4 allele (c19M4) and a neighboring SNP (c19M3) in strong disequilibrium with the ε4 polymorphism allele (see Table 2). None of the SNPs in chromosome 13 showed significant single locus associations.

TABLE 1
Allele Frequencies for Chromosome 19 and 13 Loci

| | | AD Cases | | Controls | | T-Test/ | |
|---|---|---|---|---|---|---|---|
| MARKER | ALLELE | % | (n alleles) | % | (n alleles) | CHI-SQ | PROB |
| C19M1 | C | .5227 | (440) | .4968 | (314) | 0.492 | 0.483 |
| C19M2 | A | .5959 | (438) | .6195 | (318) | 0.430 | 0.512 |
| C19M3 | T | .3144^{HWD} | (404) | .1429 | (308) | 28.167 | 0.001 |
| C19M4 | C* | .3430^{HWD} | (446) | .1171 | (316) | 50.454 | 0.001 |
| C19M5 | C | .9369 | (444) | .9263 | (312) | 0.331 | 0.565 |
| C19M6 | A | .4722 | (432) | .4810 | (316) | 0.057 | 0.812 |
| C19M7 | A | .2682 | (440) | .2803 | (314) | 0.135 | 0.714 |
| C19M8 | A | .2723 | (404) | .2930 | (314) | 0.375 | 0.540 |
| C13M1 | C | .4734 | (414) | 4902^{HWD} | (306) | 0.198 | 0.656 |
| C13M2 | C | .4726 | (438) | .5171 | (292) | 1.390 | 0.238 |
| C13M3 | A | .4953 | (422) | .4554 | (314) | 1.146 | 0.284 |
| C13M4 | C | .4048 | (420) | .4679 | (312) | 2.913 | 0.088 |
| C13M5 | A | .5920 | (424) | .5256 | (312) | 3.217 | 0.073 |

*part of APOE - ε4 allele
HWD = Genotypes significantly different from HW proportions at p < .05 level.

Hardy Weinberg Tests and Linkage Disequilibrium Strength Between the SNPs.

Tests of Hardy Weinberg equilibrium (HWE) were carried out for all loci among cases and controls separately. Significant departures from HWE are indicated in Table 1. A component of the ε4 allele and a closely linked SNP (numbers 3 and 4 in Table 1) showed significant deviation from HWE. Individuals with two copies of the ε4 allele generally have a higher risk of dementia and recessive locus effects may manifest themselves as deviations from HWE among affecteds (Nielsen et al, American Journal of Human Genetics 5, 1531-1540 (1998)).

Pairwise linkage disequilibrium values, as measured by D′ (Lewontin), were also calculated for all possible pairs of SNPs in both chromosome 19 and chromosome 13 regions among the control subjects (see Table 2). Significant linkage disequilibrium was detected (via chi-square tests) for most of the locus pairs among the 8 chromosome 19 SNPs and also among the 5 chromosome 13 SNPs (Table 2).

TABLE 2
Pairwise Linkage Disequilibrium (d'above diagonal) and Statistical Significance
(p-value, below diagonal) for the chromosome 19
and chromosome 13 SNPs.

| | | | | | | | |
|---|---|---|---|---|---|---|---|
| 1 | 2 | 3 | 4 | 5 | 6 | 7 | 8 |

Chromosome 19 (˜200-250 kb)

| | | | | | | | | |
|---|---|---|---|---|---|---|---|---|
| C19M1 | | 0.881 | 0.009 | 0.067 | 0.446 | 0.019 | 0.057 | 0.003 |
| C19M2 | <0.001 | | 0.091 | 0.115 | 0.175 | 0.016 | 0.047 | 0.1 |
| C19M3 | 0.887 | 0.093 | | 1 | 1 | 0.76 | 0.223 | 0.137 |
| C19M4 | 0.306 | 0.026 | <0.001 | | 1 | 0.602 | 0.172 | 0.236 |
| C19M5 | 0.001 | 0.300 | <0.001 | <0.001 | | 0.126 | 0.923 | 0.817 |
| C19M6 | 0.606 | 0.717 | <0.001 | <0.001 | 0.328 | | 0.146 | 0.143 |
| C19M7 | 0.356 | 0.522 | 0.041 | 0.098 | <0.001 | 0.019 | | 1 |
| C19M8 | 0.957 | 0.173 | 0.208 | 0.024 | <0.001 | 0.023 | <0.001 | |

Chromosome 13:

| | | | | | |
|---|---|---|---|---|---|
| C13M1 | | 0.01 | 0.044 | 0.185 | 0.171 |
| C13M2 | 0.801 | | 0.599 | 0.441 | 0.443 |
| C13M3 | 0.249 | <0.001 | | 1 | 1 |
| C13M4 | <0.001 | <0.001 | <0.001 | | 1 |
| C13M5 | <0.001 | <0.001 | <0.001 | <0.001 | |

Haplotype Analyses.

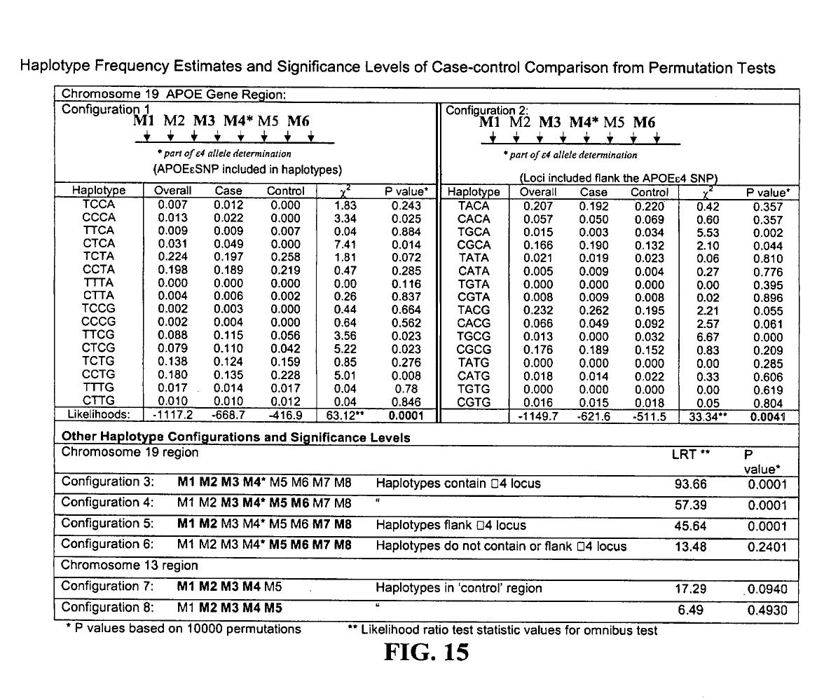

Haplotype frequencies for various marker combinations were estimated for cases and controls separately via an Expectation-Maximization algorithm (See Example 1). In FIG. 15, the table displays the results of several 4-locus estimated haplotype frequency analyses for SNPs in the chromosome 19 APOE gene region and the ‘control’ region on chromosome 13. The top two panels of the Table (FIG. 15) display haplotype frequency analysis results for two 4-locus haplotype configurations involving the APOE gene region SNPs. The first configuration (top left panel) contains SNPs C19M1, C19M3, C19M4 and C19M6, which include the two SNPs showing significant single-locus associations: the ±4 allele locus (SNP C19M4) and the neighboring locus whose allele is in strong disequilibrium with the e4 allele SNP(SNP C19M3). The second configuration (top right panel) replaces SNPs 3 and 4 with those immediately flanking them (SNPs 2 and 5) such that the haplotypes derived in this way span the same region but do not explicitly contain the significant single-locus SNPs. The 16 estimated haplotype frequencies for case and control groups are shown for both of the sets of SNPs as well as chi-square values and permutation test significance levels for frequency comparisons between the AD and control groups. The last row of the top two panels in the Table (FIG. 15) gives an “omnibus” likelihood ratio test statistic and empirically-determined (via randomization tests) significance results assessing the overall haplotype frequency profile differences between the cases and controls, rather than testing frequency differences for specific haplotypes. Note that both the configuration containing the ε4 allele and that configuration using only floating SNPs resulted in significant omnibus haplotype profile tests. This second configuration did not contain any SNPs that showed significant single locus associations (Table 1). The bottom panel of Table 2 shows the omnibus likelihood ratio test results for other 4-locus configurations in the chromosome 19 region as well as the unrelated chromosome 13 region. These results suggest that SNP combinations either directly including the ε4 locus or SNPs flanking the ε4 locus result in significantly different haplotype frequencies between cases and controls, while those combinations not containing E4 locus on the flanking SNPs (i.e., configuration 6 for ch.19 SNPs) do not show significant differences between cases and controls.

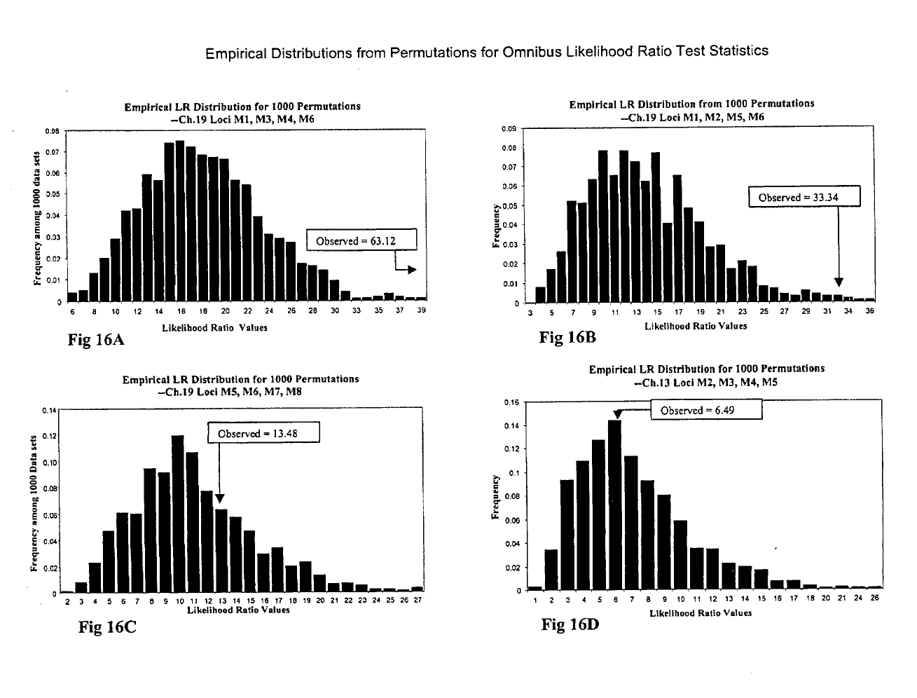

Permutation tests were used to assess the statistical significance of the haplotype frequency differences. The panels A-D of FIG. 16 display the omnibus likelihood ratio test statistic distributions for 1000 permuted data sets. As can be seen in panels A and B, the observed test statistics for haplotypes derived from sets of SNPs containing ε4 locus or flanking SNPs are extreme compared to the statistics obtained from the permutations. This suggests that there are likely to be Alzheimer's susceptibility alleles on one or some set of the chromosomes exhibiting the haplotypes studied. Panels C and D, however, show the observed statistics for a set of SNPs which do not cover the E4 locus (either within the APOE region or on chromosome 13) are not extreme (i.e., p>0.10). Thus, there is no evidence for overall haplotype frequency differences between the cases and controls with these SNP combinations.

Our results identify differences in allele and haplotype frequencies in APOE gene region variants between AD cases and non-demented controls sampled from greater France without relying on overt haplotyping through the use of relatives' genotypes, long-range PCR, or related techniques (Glaxo). Our analysis methods accommodate weak LD and potential allelic heterogeneity, since the omnibus test assesses haplotype frequency profiles rather than associations between particular haplotypes and disease status. Both weak LD among markers in a candidate region and allelic heterogeneity may result in a number of disease mutation-bearing chromosomes segregating in a population Terwilliger et al, Current Opinion in Biotechnology 9, 578-594 (1998) each with its own unique signature pattern of alleles (or haplotype). Each of these haplotypes may be greater in frequency among cases than controls but not in a pronounced way due to the number of different haplotypes of diseased individuals. Since the omnibus test assesses overall haplotype frequency profile differences rather than simple haplotype frequency differences, it can detect subtle differences between haplotypes that manifest themselves in aggregate rather than individually. Second, insignificant analysis results of anonymous markers in a non-candidate and likely inert region of the genome provide some evidence that our results with the APOE gene region are not due to stratification or an inherent statistical test bias.

Ultimately, our results suggest that the proposed genetic analysis strategy have the potential to detect linkage-disequilibrium-induced associations between anonymous SNPs and complex diseases even when the actual functional polymorphisms are not actually typed. Thus, it may be possible to systematically apply the proposed methods to identify novel disease genes underlying diseases with unknown genetic determinants.

### Example 3

### Comparison of the Accuracy of Haplotype Estimations

In addition to the simulation-based accuracy assessment detailed previously (Example 1), the results of analysis with the program of the instant invention have been compared with another E-M based estimation program, as well as to family-derived haplotype frequencies, taken as a “gold standard”. Table 3 is a table of haplotype estimation results for the program MLOCUS and for a program of the instant invention, (Schork (1999)) as well as true, family derived haplotype frequencies (i.e., from actual pedigree data). It shows results for haplotype frequency estimation for an 8-locus diallelic system. The first three columns represent 100 individuals from the CEPH data base, where the true haplotypes have been determined via family member genotypes. The three columns represent our haplotype frequency estimates, those of the MLOCUS program (Long et al, American Journal of Human Genetics 56, 799-810 (1995)), and the true frequencies based on family member data. The remaining columns represent a set of breast cancer cases and healthy controls. Haplotype frequencies were estimated for each group separately and for the combined set. The results from MLOCUS are also provided for comparison.

These results agree with our simulation-based results indicating the high accuracy of our estimation procedure. The similarity to the results of MLOCUS is expected given that they both rely on the same underlying algorithm. Some main advantages of our program versus MLOCUS, Arlequin, or other E-M based programs is the dramatically decreased computational time and the attachment of novel statistical approaches using the estimates and the final likelihoods.

TABLE 3
Haplotype Estimation Results

| Hap | | MLOCUS | SCHORK | PEDIGREE | MLOCUS | SCHORK | MLOCUS | SCHORK | MLOCUS | SCHORK |
|---|---|---|---|---|---|---|---|---|---|---|
| No. | Haplotype | CEPHGP | CEPHGP | CEPHGP | BRCAtotal | BRCAtotal | BRCAcase | BRCAcase | BRCAcon | BRCAcon |
| 1 | 11111111 | 0.29210 | 0.29208 | 0.28280 | 0.21030 | 0.21687 | 0.23710 | 0.24214 | 0.20470 | 0.20564 |
| 2 | 11222111 | 0.20090 | 0.20085 | 0.20200 | 0.02750 | 0.01921 | 0.02510 | 0.02215 | 0.02700 | 0.01695 |
| 3 | 12222111 | 0.17790 | 0.17793 | 0.17680 | 0.14840 | 0.14497 | 0.21270 | 0.20714 | 0.10000 | 0.10101 |
| 4 | 12121111 | 0.11110 | 0.11111 | 0.10610 | 0.21090 | 0.21302 | 0.20120 | 0.18571 | 0.21360 | 0.22727 |
| 5 | 21111211 | 0.09290 | 0.09293 | 0.10100 | 0.16570 | 0.16256 | 0.15240 | 0.16429 | 0.16630 | 0.15626 |
| 6 | 11111112 | 0.08590 | 0.08586 | 0.09090 | 0.01560 | 0.01479 | 0.00000 | 0.00000 | 0.01820 | 0.02020 |
| 7 | 12111111 | 0.01400 | 0.01399 | 0.01520 | 0.00000 | 0.00000 | 0.00000 | 0.00000 | 0.00000 | 0.00000 |
| 8 | 11111121 | 0.02220 | 0.02222 | 0.02520 | 0.00560 | 0.00636 | 0.00000 | 0.00000 | 0.01000 | 0.01123 |
| 9 | 21111221 | 0.00300 | 0.00303 | 0.00000 | 0.00000 | 0.00000 | 0.00000 | 0.00000 | 0.00000 | 0.00000 |
| 10 | 11221111 | 0.00000 | 0.00000 | 0.00000 | 0.18230 | 0.19231 | 0.14630 | 0.15714 | 0.20910 | 0.22042 |
| 11 | 11111211 | 0.00000 | 0.00000 | 0.00000 | 0.01870 | 0.01686 | 0.00000 | 0.00000 | 0.02630 | 0.02344 |
| 12 | 21111111 | 0.00000 | 0.00000 | 0.00000 | 0.00610 | 0.00608 | 0.00000 | 0.00000 | 0.01120 | 0.01041 |
| 13 | 21111212 | 0.00000 | 0.00000 | 0.00000 | 0.00000 | 0.00000 | 0.01220 | 0.00714 | 0.00000 | 0.00000 |
| 14 | 12111211 | 0.00000 | 0.00000 | 0.00000 | 0.00000 | 0.00000 | 0.00680 | 0.00786 | 0.00000 | 0.00000 |
| 15 | 12222211 | 0.00000 | 0.00000 | 0.00000 | 0.00000 | 0.00000 | 0.00610 | 0.00642 | 0.00000 | 0.00000 |
| 16 | 11222211 | 0.00000 | 0.00000 | 0.00000 | 0.00120 | 0.00150 | 0.00000 | 0.00000 | 0.00480 | 0.00000 |
| 17 | 11111221 | 0.00000 | 0.00000 | 0.00000 | 0.00250 | 0.00252 | 0.00000 | 0.00000 | 0.00420 | 0.00392 |
| 18 | 21121111 | 0.00000 | 0.00000 | 0.00000 | 0.00000 | 0.00000 | 0.00000 | 0.00000 | 0.00330 | 0.00000 |
| 19 | 21121211 | 0.00000 | 0.00000 | 0.00000 | 0.00260 | 0.00000 | 0.00000 | 0.00000 | 0.00130 | 0.00000 |
| 20 | 11221211 | 0.00000 | 0.00000 | 0.00000 | 0.00000 | 0.00000 | 0.00000 | 0.00000 | 0.00000 | 0.00000 |
| 21 | 12121112 | 0.00000 | 0.00000 | 0.00000 | 0.00000 | 0.00000 | 0.00000 | 0.00000 | 0.00000 | 0.00000 |
| 22 | 11212121 | 0.00000 | 0.00000 | 0.00000 | 0.00000 | 0.00000 | 0.00000 | 0.00000 | 0.00000 | 0.00000 |
| 23 | 21112211 | 0.00000 | 0.00000 | 0.00000 | 0.00000 | 0.00000 | 0.00000 | 0.00000 | 0.00000 | 0.00000 |
| 24 | 12212211 | 0.00000 | 0.00000 | 0.00000 | 0.00260 | 0.00296 | 0.00000 | 0.00000 | 0.00000 | 0.00000 |
| 25 | 11112211 | 0.00000 | 0.00000 | 0.00000 | 0.00000 | 0.00000 | 0.00000 | 0.00000 | 0.00000 | 0.00325 |
| | | 1.00000 | 1.00000 | 1.00000 | 1.00000 | 1.00001 | 0.99990 | 0.99999 | 1.00000 | 1.00000 |

### REFERENCES

Bennett, J. On the theory of random mating. Ann Eugen 18, 311-317 (1954).

Chakravarti, A. It's raining SNPs, hallelujah? Nature Genetics 19, 216-217 (1998).

Chapman, N H & Wijsman, E M, Genome screens using linkage disequilibrium tests: optimal marker characteristics and feasibility. American Journal of Human Genetics 63, 1872-1885 (1998).

Chiano M. & Clayton D., Fine Genetic Mapping Using Haplotype Analysis and the Missing Data Problem. Ann. Hum. Genet. 62, 55-60 (1998).

Clark A., Inference Of Haplotypes From PCR-Amplified Samples of Diploid Populations. Mol. Biol. Evol., 7(2), 111-122 (1990).

Clark, et al. Haplotype structure and population-genetics inferences from nucleotide-sequence variation in human lipoprotein lipase. American Journal of Human Genetics 63, 595-612 (1998).

Collins, F. S., Geyer, M. S. & Chakravarti, A. Variations on a theme: Cataloging human DNA sequence variation. Science 278, 1580-1581 (1997).

Collins, et al. New Goals for the U.S. Human Genome Projects: 1998-2003. Science 282, 682-689 (1998).

Dempster A, Laird N, & Rubin D. Maximum Likelihood From Incomplete Data Via the EM Algorithm. J. R. Stat. Soc., 39, 1-38 (1977).

Edwards, A. W. F. Likelihood, (Johns Hopkins University Press, Baltimore, 1992).

Excoffier, L. & Slatkin, M. Maximum-likelihood estimation of molecular haplotype frequencies in a diploid population. Molecular Biology and Evolution 12, 921-927 (1995).

Fallin, D. Unpublished Ph.D. Thesis Manuscript. Case Western Reserve University (1999)

Fallin, D. & Schork, N.J. The accuracy of haplotype frequency estimation involving biallelic markers and genotypic data. (in preparation) (1999).

Good, P. Permutation Tests, (Springer-Verlag, New York, 1994).

Hawley, M. E. & Kidd, K. K. HAPLO: A program using the EM algorithm to estimate the frequencies of multi-site haplotypes. The Journal of Heredity 86, 409411 (1995).

Hawley M E, Pakstis A J, & Kidd K K. A Computer Program Implementing the EM Algorithm for Haplotype Frequency Estimation. Am. J. Phys. Anthropol., S18, 104 (1994).

Hastbacka J, de la Chapelle A, Kaitila I, Sistonen P, Weaver A, Lander E. Linkage Disequilibrium Mapping In Isolated Founder Populations: Diastrophic Dysplasia in Finland. Nature Genetics, 2, 204-211 (1992).

Hill W. & Robertson A. Linkage Disequilibrium In Finite Populations. Theor. App. Genet. 38, 226-231(1968).

Hill W. Estimation of Linkage Disequilibrium in Randomly Mating Populations. Heredity, 33(2), 229-239 (1974).

Hill W. & Weir B. Maximum-likelihood estimation of gene location by linkage disequilbrium. Am J hum Genet, 54, 705-714 (1994).

Jorde L, Watkins W, Viskochil D, O'Connell P, & Ward K. Linkage Disequilibrium in the Neurofibromatosis I (NF1) region: Implications for Gene Mapping. Am. J. Hum. Genet. 53, 1038-1050 (1993).

Jorde L, Watkins W, Carlson M, Groden J, Albertsen H, Thliveris A, & Leppert M. Linkage disequilibrium predicts physical distance in the adenomatous polyposis cole region. Am J Hum Genet, 54, 884-898 (1994).

Jorde, L. Linkage Disequilibrium as a Gene-Mapping Tool. Am. J. Hum. Genet. 56, 11-14 (1995).

Kaplan, N & Weir, B. Expected behavior of conditional linkage disequilibrium. Am J Hum Genet, 51, 333-343 (1992).

Kaplan N, Hill W, & Weir B. Likelihood methods for locating disease genes in non-equilibrium populations. Am. J. Hum. Genet. 56,18-32 (1995).

Kerem B, Rommens J, Buchanan J, Markiewicz D, Cox T, Chakravarti A, Buchwald M, & Tsui L-C. Identification of the cystic fibrosis gene: Genetic analysis. Science 245, 1073-1080 (1989).

Kruglyak, L. The use of a genetic map of biallelic markers in linkage studies. Nature Genetics 17, 21-24 (1997).

Long J, Williams R, & Urbanek M. An E-M Algorithm and Testing Strategy For Multiple-Locus Haplotypes. Am. J. Hum. Genet. 56, 779-810 (1995).

Michalatos-Beloin S. et al. Molecular haplotyping of genetic markers 10 kb apart by allele-specific long-range PCR. Nucleic Acids Research 23, 48414843 (1996).

Nielsen, D M et al. Detecting marker-disease association by testing for Hardy-Weinberg disequilibrium at a marker locus. American Journal of Human Genetics 63(5), 1531-1540 (1998).

Olson J, & Wijsman E. Design and sample size considerations in the detection of linkage disequilibrium with a disease locus. Am. J. Hum. Genet. 55, 574-580 (1994).

Osier et al. Linkage disequilibrium at the ADH2 and ADH3 loci and risk of alcoholism. American Journal of Human Genetics 64, 1147-1157 (1999).

Risch, N. & Merikangas, K. The future of genetic studies of complex human diseases. Science 278, 1516-1517 (1996)

Schipper et al. Validation of haplotype frequency estimation methods. Human Immunology 59, 518-523 (1998)

Schneider et al. Arlequin ver 2000:A software for population genetics data analysis. Genetics and Biometry Laboratory University of Geneva, Switzerland (2000).

Schlesselman, J. J. Case-Control Studies, (Oxford University Press, New York, 1982).

Slatkin M, & Excoffier L. Testing for Linkage Disequilibrium in Genotypic Data Using The Expectation-Maximization Algorithm. Heredity, 76, 377-383 (1996).

Terwilliger, J. T. & Weiss, K. M. Linkage disequilibrium mapping of complex diseases: fantasy or reality? Current Opinion in Biotechnology 9, 578-594 (1998).

What is claimed is:

1. A method of determining the statistical significance of a difference between haplotype frequency profiles of at least two groups of individuals comprising:
a) determining the combined likelihood that said at least two groups of individuals are derived from the same distribution of haplotypes;
b) determining the sum of the separate likelihoods that each of said at least two groups of individuals are derived from the same distribution of haplotypes; c) determining the difference of said sum and said combined likelihood; and
d) determining the significance of this difference by simulating hypothetical groups by randomly permuting the haplotypes between groups to determine the probability that the groups do not come from the same distribution of haplotypes.

2. The method of claim 1, further comprising calculating all possible single-haplotype chi-square tests prior to determining the significance of the difference between said sum and said combined likelihood.

3. The method of claim 1, further comprising assessing the statistical significance of individual haplotypes using an odds ratio or a P-excess value.

4. A system for determining the statistical significance of the difference between haplotype frequency profiles of at least two groups of individuals, comprising:
a) first instructions for determining the combined likelihood that said at least two groups of individuals are derived from the same distribution of haplotypes;
b) second instructions for determining the sum of the separate likelihoods that each of said at least two groups of individuals are derived from the same distribution of haplotypes;
c) third instructions for determining the difference of said sum and said combined likelihood; and
d) fourth instructions for determining the significance of this difference by simulating hypothetical groups by randomly permuting the haplotypes between groups to determine the probability that the groups do not come from the same distribution of haplotypes.

5. The system of claim 4, further comprising fifth instructions for calculating all possible single-haplotype chi-square tests prior to determining the significance of the difference between said sum and said combined likelihood.

6. The system of claim 4, further comprising fifth instructions for assessing the statistical significance of individual haplotypes using an odds ratio or a P-excess value.

7. A programmed storage device comprising instructions that when executed perform a method comprising:
a) determining the statistical significance of the difference between haplotype frequency profiles of at least two groups of individuals by comparing the final likelihood that all groups of individuals come from the same distribution of haplotypes with the sum of the final likelihoods for each group separately; and
b) determining the significance of this difference by simulating hypothetical groups by randomly permuting the haplotypes between groups to determine the probability that the groups do not come from the same distribution of haplotypes.

8. The programmed storage device of claim 7, further comprising instructions that when executed perform a method of calculating all possible single-haplotype chi-square tests prior to determining the significance of the difference between said sum and said combined likelihood.

9. The programmed storage device of claim 7, further comprising instructions that when executed perform a method of assessing the statistical significance of individual haplotypes using an odds ratio or a P-excess value.

10. A method of estimating haplotype frequencies for single nucleotide polymorphisms in groups of individuals comprising:
a) estimating all haplotype and diplotype probabilities for said groups of individuals using an estimation-maximization process;
b) storing said probabilities; and
c) repeating said estimation-maximization process using random starting values.

11. The method of claim 10, wherein all haplotypes are coded with binary mask arrays, and wherein identical genotypes are grouped prior to performing said estimations.

12. A computer system for estimating haplotype frequencies for single nucleotide polymorphisms in groups of individuals comprising:
a) first instructions that when executed perform a method of estimating all haplotype and diplotype probabilities using an estimation-maximization process;
b) second instructions that when executed perform a method of storing said haplotype and diplotype probabilities; and
c) third instructions that when executed perform said estimation-maximization process that is automatically repeated using random starting values.

13. The computer system of claim 12, wherein all haplotypes are coded with binary mask arrays, and wherein identical genotypes are grouped prior to performing estimations.

14. A programmed storage device comprising estimation-maximization instructions that when executed perform the method of:
a) estimating haplotype frequencies for single nucleotide polymorphisms in groups of individuals comprising estimating and storing all haplotype and diplotype probabilities using an estimation-maximization process; and
b) repeating said estimation-maximization process using random starting values.

15. The programmed storage device of claim 14, wherein all haplotypes are coded with binary mask arrays, and wherein identical genotypes are grouped prior to performing estimations.

16. A method of determining the statistical significance of the difference between haplotype frequency profiles of at least two groups of individuals, comprising:
a) estimating haplotype frequencies using single nucleotide polymorphism data for each group individually and for each group in combination with another group, wherein all haplotype and diplotype probabilities are calculated once and then stored, and wherein a maximization process is automatically repeated for each group using random starting values in order to determine final likelihoods;
b) comparing the final likelihood that all groups come from the same distribution of haplotypes with the sum of the final likelihoods for each group separately to determine their difference; and
c) determining the significance of this difference by simulating hypothetical groups by randomly permuting the haplotypes between groups to determine the probability that the groups do not come from the same distribution of haplotypes.

17. The method of claim 16, wherein all haplotypes are coded with binary mask arrays, and wherein identical genotypes are grouped prior to performing operations.

18. A system for determining the statistical significance of the difference between haplotype frequency profiles of at least two groups of individuals, comprising:
a) a first module configured to estimate haplotype frequencies using single nucleotide polymorphism data for each group individually and for each group in combination with another group, wherein all haplotype and diplotype probabilities are calculated once and then stored, and wherein the maximization process is automatically repeated for each group using random starting values, to determine final likelihoods;
b) a second module configured to compare the final likelihood that all groups come from the same distribution of haplotypes with the sum of the final likelihoods for each group separately to determine their difference; and
c) a third module configured to determine the significance of this difference by simulating hypothetical groups by randomly permuting the haplotypes between groups to determine the probability that the groups do not come from the same distribution of haplotypes.

19. The system of claim 18, wherein all haplotypes are coded with binary mask arrays, and wherein identical genotypes are grouped prior to performing estimations.

20. A programmed storage device comprising instructions that when executed perform a method of determining the statistical significance of the difference between haplotype frequency profiles of at least two groups of individuals, comprising
a) a first module adapted to perform a method of estimating haplotype frequencies using single nucleotide polymorphism data for each group individually and for each group in combination with the other group, wherein all haplotype and diplotype probabilities are calculated once and then stored, and wherein the maximization process is automatically repeated for each group using random starting values to determine final likelihoods;
b) a second module adapted to compare the final likelihood that all groups come from the same distribution of haplotypes with the sum of the final likelihoods for each group separately to determine their difference; and
c) a third module adapted to determine the significance of this difference by simulating hypothetical groups by randomly permuting the haplotypes between groups to determine the probability that the groups do not come from the same distribution of haplotypes.

21. The programmed device of claim 20, wherein all haplotypes are coded with binary mask arrays, and wherein identical genotypes are grouped prior to performing estimations.

22. A method of determining an association between a haplotype and a phenotype, comprising:
a) estimating haplotype frequencies using single nucleotide polymorphism data for an affected group and an unaffected group individually and in combination with another group, wherein all haplotype and diplotype probabilities are calculated once and then stored, and wherein a maximization process is automatically repeated for each group using random starting values to determine final likelihoods;
b) comparing the final likelihood that both groups come from the same distribution of haplotypes with the sum of the final likelihoods for each group separately to determine their difference; and
c) determining the significance of this difference by simulating hypothetical groups by randomly permuting the haplotypes between groups to determine the probability that the groups do not come from the same distribution of haplotypes and determine whether a statistically significant association exists between said haplotype and said phenotype.

23. A method of determining an association between a haplotype and a phenotype, comprising:
a) estimating haplotype frequencies using single nucleotide polymorphism data for an affected group and an unaffected group individually and in combination with another group, wherein all haplotype and diplotype probabilities are calculated once;
b) storing said probabilities; and
c) repeating a maximization process for each group using random starting values to determine whether a statistically significant association exists between said haplotype and said phenotype.

24. A method of detecting an association between a haplotype and a phenotype, comprising:
a) comparing a final likelihood that members of an affected group and an unaffected group come from the same distribution of haplotypes with the sum of the final likelihoods for each of said groups separately to determine their difference; and
b) determining the significance of this difference by simulating hypothetical groups by randomly permuting the haplotypes between groups to determine the probability that the groups do not come from the same distribution of haplotypes and whether a statistically significant association exists between said haplotype and said phenotype.

25. A system for detecting an association between a haplotype and a phenotype, comprising:
a) first instructions for estimating haplotype frequencies using single nucleotide polymorphism data for an affected group and an unaffected group individually and in combination, wherein all haplotype and diplotype probabilities are calculated once, and wherein the maximization process is automatically repeated using random starting values to determine final likelihoods;
b) second instructions for comparing the final likelihood that both groups come from the same distribution of haplotypes with the sum of the final likelihoods for each group separately; and
c) third instructions for determining the significance of this difference by simulating hypothetical groups by randomly permuting the haplotypes between groups to determine the probability that the groups do not come from the same distribution of haplotypes and determine whether a statistically significant association exists between said haplotype and said phenotype.

26. A system for detecting an association between a haplotype and a phenotype, comprising:
a) instructions for estimating haplotype frequencies using single nucleotide polymorphism data for an affected and an unaffected group individually and in combination, wherein all haplotype and diplotype probabilities are calculated once; and
b) repeating a maximization process using random starting values to determine whether a statistically significant association exists between said haplotype and said phenotype.

27. A system for detecting an association between a haplotype and a phenotype, comprising:
a) first instructions for comparing the final likelihood that the members of an affected and an unaffected group come from the same distribution of haplotypes with the sum of the final likelihoods for each group separately; and
b) second instructions for determining the significance of this difference by simulating hypothetical groups by randomly permuting the haplotypes between groups to determine the probability that the groups do not come from the same distribution of haplotypes and whether a statistically significant association exists between said haplotype and said phenotype.

28. A programmed storage device comprising instructions that when executed perform a method of detecting an association between a haplotype and a phenotype, comprising:
a) estimating haplotype frequencies using single nucleotide polymorphism data for an affected and an unaffected group individually and in combination, wherein all haplotype and diplotype probabilities are calculated once and are stored, and wherein the maximization process is automatically repeated using random starting values to determine final likelihoods;
b) comparing the final likelihood that both groups come from the same distribution of haplotypes with the sum of the final likelihoods for each group separately; and
c) determining the significance of this difference by simulating hypothetical groups by randomly permuting the haplotypes between groups to determine the probability that the groups do not come from the same distribution of haplotypes and determine whether a statistically significant association exists between said haplotype and said phenotype.

29. A programmed storage device comprising instructions that when executed perform a method of detecting an association between a haplotype and a phenotype, comprising:
a) estimating haplotype frequencies using single nucleotide polymorphism data for an affected and an unaffected group individually and in combination, wherein all haplotype and diplotype probabilities are calculated once; and
b) repeating a maximization process using random starting values to determine whether a statistically significant association exists between said haplotype and said phenotype.

30. A programmed storage device comprising instructions that when executed perform a method of detecting an association between a haplotype and a phenotype, comprising:
a) comparing a likelihood that members of an affected group and an unaffected group come from the same distribution of haplotypes with the sum of the final likelihoods for each group separately;
b) determining the significance of this difference by simulating hypothetical groups by randomly permuting the haplotypes between groups to determine the probability that the groups do not come from the same distribution of haplotypes; and
c) determining whether a statistically significant association exists between said haplotype and said phenotype.

31. A computer-readable data signal embedded in a transmission medium that when executed performs a method of determining the statistical significance of the difference between haplotype frequency profiles of at least two groups of individuals, comprising:
a) code segments comparing the final likelihood that all groups come from the same distribution of haplotypes with the sum of the final likelihoods for each group separately; and
b) code segments determining the significance of this difference by simulating hypothetical groups by randomly permuting the haplotypes between groups to determine the probability that the groups do not come from the same distribution of haplotypes.

32. A wide area computer network for determining the statistical significance of the difference between haplotype frequency profiles of at least two groups of individuals, comprising:
a) a server comprising single nucleotide polymorphism data; and
b) a workstation comprising instructions for estimating haplotype frequencies using said nucleotide polymorphism data for each group individually and in combination with the other group, wherein all haplotype and diplotype probabilities are calculated once and are stored, and wherein the maximization process is automatically repeated using random starting values.

33. The wide area computer network of claim 32, wherein said network comprises the Internet.

34. The wide area computer network of claim 32, wherein said instructions are stored in a memory.

35. The wide area computer network of claim 32, wherein said instructions are stored in a code segment.

36. A computer-readable data signal embedded in a transmission medium that when interpreted performs a method determining the statistical significance of the difference between haplotype frequency profiles of at least two groups of individuals, comprising:
a) first signals adapted to perform a method of estimating haplotype frequencies using single nucleotide polymorphism data for each group individually and in combination with the other group, wherein all haplotype and diplotype probabilities are calculated once and are stored, and wherein a maximization process is automatically repeated using random starting values, to determine final likelihoods;
b) second signals adapted to compare the final likelihood that all groups come from the same distribution of haplotypes with the sum of the final likelihoods for each group separately; and
c) third signals adapted to determine the significance of this difference by simulating hypothetical groups by randomly permuting the haplotypes between groups to determine the probability that the groups do not come from the same distribution of haplotypes.

37. A computer system for detecting an association between a haplotype and a phenotype, comprising:
a) a first code segment configured to estimate haplotype frequencies using single nucleotide polymorphism data for an affected and an unaffected group individually and in combination, wherein all haplotype and diplotype probabilities are calculated once and are stored, and wherein a maximization process is automatically repeated using random starting values to determine final likelihoods;
b) a second code segment configured to compare the final likelihood that both groups come from the same distribution of haplotypes with the sum of the final likelihoods for each group separately; and
c) a third code segment configured to determine the significance of this difference by simulating hypothetical groups by randomly permuting the haplotypes between groups to determine the probability that the groups do not come from the same distribution of haplotypes and determine whether a statistically significant association exists between said haplotype and said phenotype.

38. A computer-readable data signal embedded in a transmission medium that when executed performs a method of detecting an association between a haplotype and a phenotype, comprising:
a) a first signal for estimating haplotype frequencies using single nucleotide polymorphism data for an affected and an unaffected group individually and in combination, wherein all haplotype and diplotype probabilities are calculated once and are stored; and
b) a second signal for repeating a maximization process using random starting values to determine whether a statistically significant association exists between said haplotype and said phenotype.

39. A wide area computer system for detecting an association between a haplotype and a phenotype, comprising:
a) a first memory comprising first code segments adapted to compare the final likelihood that the members of an affected and an unaffected group come from the same distribution of haplotypes with the sum of the final likelihoods for each group separately;
b) a second memory comprising second code segments adapted to determine the significance of this difference by simulating hypothetical groups by randomly permuting the haplotypes between groups to determine the probability that the groups do not come from the same distribution of haplotypes and whether a statistically significant association exists between said haplotype and said phenotype.
